(19) United States
(12) Patent Application Publication
(10) Pub. No.: US 2023/0172901 A1
(43) Pub. Date: Jun. 8, 2023

(54) COMPOSITIONS AND METHODS FOR THE PROPHYLAXIS AND TREATMENT OF FIBROTIC AND INFLAMMATORY CONDITIONS

(71) Applicant: GRETALS AUSTRALIA PTY LTD, Mornington (AU)

(72) Inventors: Alistair Cumming, Mornington (AU); Kenneth Snibson, Mornington (AU)

(21) Appl. No.: 17/923,782

(22) PCT Filed: May 7, 2021
(86) PCT No.: PCT/AU2021/050421

§ 371 Date: Nov. 7, 2022

(30) Foreign Application Priority Data
May 8, 2020 (AU) 2020901475
Dec. 29, 2020 (AU) 2020904863

(51) Int. Cl. A61K 31/353; A61P 17/02; A61P 11/00

(52) U.S. Cl. CPC A61K 31/353; A61P 17/02; A61P 11/00

(57) ABSTRACT

The present invention relates to the prevention and/or treatment of a fibrotic or inflammatory condition by the administration of a compound to an animal in need thereof. In particular, flavonones extracted from natural plant materials such as pinocembrin (5,7-dihydroxy-2-phenyl-2,3-dihydro-4h-chromen-4-one) have been found to be useful for airway conditions having fibrotic and inflammatory components.

### FIELD OF THE INVENTION

The present invention relates generally to the field of human and veterinary medicine. In particular, the invention relates to the prevention and/or treatment of a fibrotic or inflammatory condition by the administration of a compound to an animal in need thereof.

### BACKGROUND TO THE INVENTION

Fibrosis is a pathological outcome that may result from the wound healing response to a tissue injury. In some instances, fibrosis is caused by an unknown mechanism, and in such cases is typically known as an idiopathic fibrosis. A prominent example is idiopathic pulmonary fibrosis (IPF). It remains possible of course that an idiopathic fibrosis results from an undetected tissue injury and subsequent wound healing response.

It is known that wound healing comprises the sequential phases of injury, inflammation and repair. Whilst wound healing is clearly necessary in maintaining the integrity and proper functioning of the body, the formation of fibrotic scar tissue can lead to serious health consequences. In the case of IPF, a dramatic decrease in lung function is often seen which in many cases leads to death of the patient.

The injury which triggers wound healing may be caused by one or more of physical trauma, autoimmune reactions, infection (bacterial, viral or otherwise), and exposure to foreign bodies. Where the injured tissue comprises endothelial cells, mediators of inflammation are released which in turn modulate the coagulation pathways leading to the formation of a fibrin clot to prevent blood loss. In IPF, lung tissues are noted to contain elevated levels of platelet-differentiating factor and x-box-binding protein, indicating that clotting pathways are continuously activated.

In addition, thrombin is detected in the lungs of IPF patients and also sufferers of other pulmonary fibrotic conditions. Thrombin is a participant in coagulation pathways leading to the formation of fibrin clots, and also causes proliferation of fibroblasts and differentiation into myofibroblasts.

Damage to lung epithelium can lead to similar triggering of fibrin formation and also leads interstitial edema, localised acute inflammation and separation of epithelial cells from the basement membrane.

Matrix metalloproteinases (MMPs) regulate the passage of inflammatory cells into and out of areas of damage, with MMP inhibitors modulating the process. The balance between MMPs and their inhibitors regulate inflammation and determine the net amount of collagen deposited during the healing response.

The inflammatory phase commences with chemokines attracting lymphocytes, neutrophils, eosinophils and macrophages. It is thought that phagocytic macrophages recruited in the later periods of the inflammatory response may assist in the clearance of fibroblasts thereby promoting normal healing and avoiding pathological fibrosis.

In the repair phase of wound healing, a fibrin-rich scaffold forms followed by wound contraction, closure and re-epithelialisation. So-called granulation tissue is formed by the association of the fibrin scaffold with fibronectin, smooth muscle actin and collagens. Fibroblasts and alveolar macrophages obtained from IPF patients display elevated levels smooth muscle actin and fibronectin suggesting an unusually high level of fibroblast activation.

The depletion of inflammatory cells (and especially myofibroblasts) is important in halting collagen deposition. In IPF patients, the depletion of fibroblasts can be delayed, possibly due to a resistance to apoptotic signals. It has been proposed that resistance to apoptosis is the underlying mechanism to the fibrotic disease, however, some studies show elevated rates of collagen-secreting fibroblasts and epithelial cell apoptosis in IPF, suggesting that other factors are involved.

In a broad sense fibrosis is the development of excessive amounts of connective tissue in the body, formed by a normal or abnormal wound healing response. The net result is the formation of scar tissue which can be either beneficial (for example closure of a wound) or deleterious to health such as in IPF, or other fibrosis-related conditions including cystic fibrosis, myocardial fibrosis, Peyronie's disease, and scleroderma.

The prior art provides a number of treatments for fibrotic conditions, however each presents one or more disadvantages. For example, lung transplant is an option for IPF patients however the shortage of donor organs and the need for immunosuppression place significant limitation on that mode of treatment. Pharmaceutical compounds such as Nintendanib (Ofev™ Boehringer Ingelheim) can improve quality of life by improving respiratory parameters, but do not improve survival. As another example, Perfenidone (Esbriet™, Genetech) has been found to improve progression-free survival, however the drug provokes a range of side effects in the skin, gastrointestinal tract, liver, and nervous system.

The search for improved or alternative means for treating pulmonary fibrosis has become particularly urgent in this era of the SARS-CoV-2 pandemic. As mentioned supra, infection can trigger an injury which in turn leads to the development of fibrosis of the affected tissue or organ. One study has reported that 17% of COVID-19 patients exhibited fibrous stripes by chest CT scans, and proposed that the fibrous lesions form in the course of healing of pulmonary chronic inflammation or proliferative diseases, with gradual replacement of cellular components of the infected lung tissue by scar tissue. Thus, while effective treatments may be developed to clear the SARS-CoV-2 infection from the lungs, chronic health issues in the form of pulmonary fibrosis may nevertheless remain.

While inflammation may be a component of fibrosis, it is a process which on its own may lead to a range of conditions including pulmonary inflammation, dermal inflammation, gastrointestinal inflammation, autoimmune diseases, urinary system diseases, sarcoidosis, transplant rejection, vasculitis, atherosclerosis, pelvic inflammatory disease, rheumatic fever, and otitis. The prior art teaches the use of various pharmaceutical substances such as corticosteroids, dexamethasone, and biologics (such as antibody therapy), however each presents undesirable side effects.

It is an aspect of the present invention to provide an improvement to compositions and methods for the prophylaxis and treatment of fibrosis-related and inflammation-related conditions, and particularly those conditions having involvement of lung tissue. It is a further aspect of the present invention to provide a useful alternative to prior art methods and compositions for prophylaxis and treatment of fibrosis-related and inflammation-related conditions, and particularly those conditions having involvement of lung tissue.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect, but not necessarily the broadest aspect, the present invention provides a method for the treatment and/or prophylaxis of a fibrotic or inflammatory condition, the method comprising the administration of an effective amount of a flavonoid to an animal in need thereof.

In one embodiment of the first aspect, the fibrotic condition is caused at least in part by a wound healing response.

In one embodiment of the first aspect, the wound healing response occurs in a tissue comprising epithelial and/or endothelial cells.

In one embodiment of the first aspect, the fibrotic condition is selected from the group consisting of: pulmonary fibrosis (including idiopathic pulmonary fibrosis, infection-induced pulmonary fibrosis, radiation-induced pulmonary fibrosis, progressive massive fibrosis, cystic fibrosis), pancreatic fibrosis (including cystic fibrosis), retropertinoneal fibrosis, arterial fibrosis (including arterial stiffness), intestinal fibrosis (including Crohn's disease), joint fibrosis (including athrofibrosis of the knee, shoulder and other joints, adhesive capsulitis), manual/digital fibrosis (including Dupuytren's contracture), dermal fibrosis (including keloid, nephrogenic systemic fibrosis, scleroderma), penis (including Peyronie's disease), lymph node fibrosis (including mediastinal fibrosis) and myocardial fibrosis (including interstitial fibrosis and replacement fibrosis).

In one embodiment of the first aspect, the fibrotic condition is a pulmonary fibrosis, and the inflammatory condition is a pulmonary inflammation.

In one embodiment of the first aspect, the flavonoid is a flavanone.

In one embodiment of the first aspect, the flavanone has a chemical structure according to formula 1:

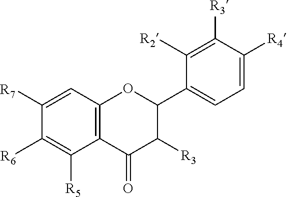

wherein R2′, R3, R3′, R4′, R5, R6, R7 are each independently:
H,
OH,
O—,
O—CH3,
a glucoside (including a rhamnosidoglucoside), or
any other organic functional group.

In one embodiment of the first aspect, R2′, R3, R3′, R4′, R5, R6, R7 are as follows:

| R3 | R5 | R6 | R7 | R2′ | R3′ | R4′ | Name |
|---|---|---|---|---|---|---|---|
| H | H | H | H | H | H | H | Flavanone |
| H | OCH₃ | H | H | H | H | H | 5-Methoxyflavanone |
| H | H | OH | H | H | H | H | 6-Hydroxyflavanone |
| H | H | OCH₃ | H | H | H | H | 6-Methoxyflavanone |
| H | H | H | OH | H | H | H | 7-Hydroxyflavanone |
| H | H | H | H | OH | H | H | 2′-Hydroxyflavanone |
| H | H | H | H | H | H | OH | 4′-Hydroxyflavanone |
| H | H | H | H | H | H | OCH3 | 4′-Methoxyflavanone |
| H | OH | H | OH | H | H | H | Pinocembrin |
| H | OH | H | OCH₃ | H | H | H | Pinocembrin-7-methylether |
| H | OH | H | OH | H | H | OH | Naringenin |
| H | OH | H | OH | H | H | OCH3 | Isosakuranetin |
| H | OH | H | OCH3 | H | H | OH | Sakuranetin |
| H | OH | H | Gla | H | H | OH | Naringenin-7-glucoside |
| H | OH | H | Rh-Glb | H | H | OH | Naringin |
| H | OH | H | OH | H | OH | OH | Eriodictyol |
| H | OH | H | OH | H | OCH₃ | OH | Homoeriodictyol |
| H | OH | H | OH | H | OH | OCH₃ | Hesperetin |
| OH | OH | H | OH | H | OH | OH | Taxifolin |

a; Gl = Glucoside.
b; Rh-Gl = Rhamnosidoglucoside.

In one embodiment of the first aspect, the flavanone is dihydroxyflavanone and/or a (2S)-flavan-4-one, or a functional derivative thereof.

In one embodiment of the first aspect, the flavanone is (2S)-5,7-dihydroxy-2-phenyl-2,3-dihydrochromen-4-one, or a functional derivative thereof.

In one embodiment of the first aspect, the flavonoid is of the type naturally synthesized in a plant cell, although is not necessarily obtained from a plant cell for use in the method.

In one embodiment of the first aspect, use of the flavonoid in a sheep model of lung disease results in an improvement in any one or mode of lung function, presence of neutrophils and/or inflammatory cells in a lung lavage fluid, histologically assessed inflammation and/or fibrosis.

In one embodiment of the first aspect, sheep model of lung disease relies on bleomycin-induced lung damage.

In one embodiment of the first aspect, the flavonoid is delivered directly to the tissue having fibrosis, potentially having fibrosis or predicted to have fibrosis in the future.

In one embodiment of the first aspect, the flavonoid is delivered directly to the lungs.

In one embodiment of the first aspect, the flavonoid is formulated as an inhalable powder or a solution deliverable by a nebulizer, or a solution deliverable by a biopsy port of a bronchoscope.

In a second aspect, the present invention provides the use of a flavonoid for the prophylaxis or treatment of a fibrotic or inflammatory condition.

In one embodiment of the second aspect, the fibrotic condition and/or the inflammatory condition is caused at least in part by a wound healing response, or exposure of an environmental agent including an allergen.

In one embodiment of the second aspect, the wound healing response or exposure to the environmental agent occurs in a tissue comprising epithelial and/or endothelial cells.

In one embodiment of the second aspect, the fibrotic condition is selected from the group consisting of: pulmonary fibrosis (including idiopathic pulmonary fibrosis, infection-induced pulmonary fibrosis, radiation-induced pulmonary fibrosis, progressive massive fibrosis, cystic fibrosis), pancreatic fibrosis (including cystic fibrosis), retropertinoneal fibrosis, arterial fibrosis (including arterial stiffness), intestinal fibrosis (including Crohn's disease), joint fibrosis (including athrofibrosis of the knee, shoulder and other joints, adhesive capsulitis), manual/digital fibrosis (including Dupuytren's contracture), dermal fibrosis (including keloid, nephrogenic systemic fibrosis, scleroderma), penis (including Peyronie's disease), lymph node fibrosis (including mediastinal fibrosis) and myocardial fibrosis (including interstitial fibrosis and replacement fibrosis), and the inflammatory condition is selected from the group consisting of: pulmonary inflammation (including COPD, asthma, rhinitis, bronchitis), dermal inflammation (including acne and scleroderma), gastrointestinal inflammation (including celiac disease, Crohn's disease, colitis, diverticulitis), autoimmune diseases (such as SLE), urinary system diseases (including glomerulonephritis, cystitis, protastitis), sarcoidosis, transplant rejection, vasculitis, atherosclerosis, pelvic inflammatory disease, rheumatic fever, and otitis.

In one embodiment of the second aspect, the fibrotic condition is a pulmonary fibrosis, and the inflammatory condition is a pulmonary inflammation.

In one embodiment of the second aspect, the flavonoid is a flavanone.

In one embodiment of the second aspect, the flavanone has a chemical structure according to formula 1:

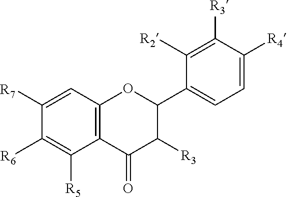

wherein R2′, R3, R3′, R4′, R5, R6, R7 are each independently:
H,
OH,
O—,
O—CH3,
a glucoside (including a rhamnosidoglucoside), or
any other organic functional group.

In one embodiment of the second aspect, R2′, R3, R3′, R4′, R5, R6, R7 are as follows:

| R3 | R5 | R6 | R7 | R2′ | R3′ | R4′ | Name |
|---|---|---|---|---|---|---|---|
| H | H | H | H | H | H | H | Flavanone |
| H | OCH₃ | H | H | H | H | H | 5-Methoxyflavanone |
| H | H | OH | H | H | H | H | 6-Hydroxyflavanone |
| H | H | OCH₃ | H | H | H | H | 6-Methoxyflavanone |
| H | H | H | OH | H | H | H | 7-Hydroxyflavanone |
| H | H | H | H | OH | H | H | 2′-Hydroxyflavanone |
| H | H | H | H | H | H | OH | 4′-Hydroxyflavanone |
| H | H | H | H | H | H | OCH3 | 4′-Methoxyflavanone |
| H | OH | H | OH | H | H | H | Pinocembrin |
| H | OH | H | OCH₃ | H | H | H | Pinocembrin-7-methylether |
| H | OH | H | OH | H | H | OH | Naringenin |
| H | OH | H | OH | H | H | OCH3 | Isosakuranetin |
| H | OH | H | OCH3 | H | H | OH | Sakuranetin |
| H | OH | H | Gla | H | H | OH | Naringenin-7-glucoside |
| H | OH | H | Rh-Glb | H | H | OH | Naringin |
| H | OH | H | OH | H | OH | OH | Eriodictyol |
| H | OH | H | OH | H | OCH₃ | OH | Homoeriodictyol |
| H | OH | H | OH | H | OH | OCH₃ | Hesperetin |
| OH | OH | H | OH | H | OH | OH | Taxifolin |

In one embodiment of the second aspect, the flavanone is dihydroxyflavanone and/or a (2S)-flavan-4-one, or a functional derivative thereof.

In one embodiment of the second aspect, the flavanone is (2S)-5,7-dihydroxy-2-phenyl-2,3-dihydrochromen-4-one, or a functional derivative thereof.

In one embodiment of the second aspect, the flavonoid is of the type naturally synthesized in a plant cell, although is not necessarily obtained from a plant cell for use in the method. In one embodiment, the flavonoid is in a racemic form in which case it may be obtained from a fermentation process.

In one embodiment of the second aspect, use of the flavonoid in a sheep model of lung disease results in an improvement in any one or mode of lung function, presence of neutrophils and/or inflammatory cells in a lung lavage fluid, histologically assessed inflammation and/or fibrosis.

In one embodiment of the second aspect, the sheep model of lung disease relies on bleomycin-induced lung damage.

In one embodiment of the second aspect, the flavonoid is delivered directly to the tissue having fibrosis, potentially having fibrosis or predicted to have fibrosis in the future.

In one embodiment of the second aspect, the flavonoid is delivered directly to the lungs and/or the airways.

In one embodiment of the second aspect, the flavonoid is formulated as an inhalable powder or a solution deliverable by a nebulizer, or a solution deliverable by a biopsy port of a bronchoscope.

In a third aspect, the present invention provides the use of a flavonoid in the manufacture of a medicament for the treatment of a fibrotic or inflammatory condition.

In one embodiment of the third aspect, the fibrotic condition is caused at least in part by a wound healing response.

In one embodiment of the third aspect, the wound healing response occurs in a tissue comprising epithelial and/or endothelial cells.

In one embodiment of the third aspect, the fibrotic condition is selected from the group consisting of: pulmonary fibrosis (including idiopathic pulmonary fibrosis, infection-induced pulmonary fibrosis, radiation-induced pulmonary fibrosis, progressive massive fibrosis, cystic fibrosis), pancreatic fibrosis (including cystic fibrosis), retropertinoneal fibrosis, arterial fibrosis (including arterial stiffness), intestinal fibrosis (including Crohn's disease), joint fibrosis (including athrofibrosis of the knee, shoulder and other joints, adhesive capsulitis), manual/digital fibrosis (including Dupuytren's contracture), dermal fibrosis (including keloid, nephrogenic systemic fibrosis, scleroderma), penis (including Peyronie's disease), lymph node fibrosis (including mediastinal fibrosis) and myocardial fibrosis (including interstitial fibrosis and replacement fibrosis), and the inflammatory condition is selected from the group consisting of: pulmonary inflammation (including COPD, asthma, rhinitis, bronchitis), dermal inflammation (including acne and scleroderma), gastrointestinal inflammation (including celiac disease, Crohn's disease, colitis, diverticulitis), autoimmune diseases (such as SLE), urinary system diseases (including glomerulonephritis, cystitis, protastitis), sarcoidosis, transplant rejection, vasculitis, atherosclerosis, pelvic inflammatory disease, rheumatic fever, and otitis.

In one embodiment of the third aspect, the fibrotic condition is a pulmonary fibrosis, and the inflammatory condition is a pulmonary inflammation.

In one embodiment of the third aspect, the flavonoid is a flavanone.

In one embodiment of the third aspect, the flavanone has a chemical structure according to formula 1:

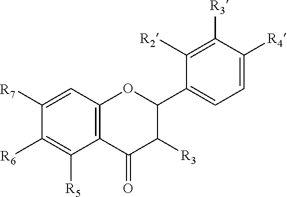

wherein R2′, R3, R3′, R4′, R5, R6, R7 are each independently:
H,
OH,
O—,
O—CH3,
a glucoside (including a rhamnosidoglucoside), or
any other organic functional group.

In one embodiment of the third aspect, R2′, R3, R3′, R4′, R5, R6, R7 are as follows:

| R3 | R5 | R6 | R7 | R2′ | R3′ | R4′ | Name |
|---|---|---|---|---|---|---|---|
| H | H | H | H | H | H | H | Flavanone |
| H | OCH₃ | H | H | H | H | H | 5-Methoxyflavanone |
| H | H | OH | H | H | H | H | 6-Hydroxyflavanone |
| H | H | OCH₃ | H | H | H | H | 6-Methoxyflavanone |
| H | H | H | OH | H | H | H | 7-Hydroxyflavanone |
| H | H | H | H | OH | H | H | 2′-Hydroxyflavanone |
| H | H | H | H | H | H | OH | 4′-Hydroxyflavanone |
| H | H | H | H | H | H | OCH3 | 4′-Methoxyflavanone |
| H | OH | H | OH | H | H | H | Pinocembrin |
| H | OH | H | OCH₃ | H | H | H | Pinocembrin-7-methylether |
| H | OH | H | OH | H | H | OH | Naringenin |
| H | OH | H | OH | H | H | OCH3 | Isosakuranetin |
| H | OH | H | OCH3 | H | H | OH | Sakuranetin |
| H | OH | H | Gla | H | H | OH | Naringenin-7-glucoside |
| H | OH | H | Rh-Glb | H | H | OH | Naringin |
| H | OH | H | OH | H | OH | OH | Eriodictyol |
| H | OH | H | OH | H | OCH₃ | OH | Homoeriodictyol |
| H | OH | H | OH | H | OH | OCH₃ | Hesperetin |
| OH | OH | H | OH | H | OH | OH | Taxifolin |

In one embodiment of the third aspect, the flavanone is dihydroxyflavanone and/or a (2S)-flavan-4-one, or a functional derivative thereof.

In one embodiment of the third aspect, the flavanone is (2S)-5,7-dihydroxy-2-phenyl-2,3-dihydrochromen-4-one, or a functional derivative thereof.

In one embodiment of the third aspect, the flavonoid is of the type naturally synthesized in a plant cell, although is not necessarily obtained from a plant cell for use in the method.

In one embodiment of the third aspect, use of the flavonoid in a sheep model of lung disease results in an improvement in any one or mode of lung function, presence of neutrophils and/or inflammatory cells in a lung lavage fluid, histologically assessed inflammation and/or fibrosis.

In one embodiment of the third aspect, the sheep model of lung disease relies on bleomycin-induced lung damage.

In one embodiment of the third aspect, the flavonoid is delivered directly to the tissue having fibrosis and/or inflammation, potentially having fibrosis and/or inflammation or predicted to have fibrosis and/or inflammation in the future.

In one embodiment of the third aspect, the flavonoid is delivered directly to the lungs and/or the airways.

In one embodiment of the third aspect, the flavonoid is formulated as an inhalable powder or a solution deliverable by a nebulizer, or a solution deliverable by a biopsy port of a bronchoscope.

In a fourth aspect, the present invention provides a pharmaceutical composition comprising a flavonoid, the composition being formulated so as to be suitable for delivery to the lungs and/or airways of an animal.

In one embodiment of the fourth aspect, the pharmaceutical composition is formulated so as to be suitable for direct delivery to the lungs and/or airways of an animal via the animal's airways.

In one embodiment of the fourth aspect, the pharmaceutical composition is formulated as an inhalable powder or a solution deliverable by a nebulizer, or a solution deliverable by a biopsy port of a bronchoscope.

In one embodiment of the fourth aspect, the flavonoid is a flavanone.

In one embodiment of the fourth aspect, the flavanone has a chemical structure according to formula 1:

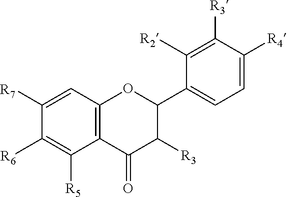

wherein R2′, R3, R3′, R4′, R5, R6, R7 are each independently:
H,
OH,
O—,
O—CH3,
a glucoside (including a rhamnosidoglucoside), or
any other organic functional group.

In one embodiment of the fourth aspect, R2′, R3, R3′, R4′, R5, R6, R7 are as follows:

| R3 | R5 | R6 | R7 | R2′ | R3′ | R4′ | Name |
|---|---|---|---|---|---|---|---|
| H | H | H | H | H | H | H | Flavanone |
| H | OCH₃ | H | H | H | H | H | 5-Methoxyflavanone |
| H | H | OH | H | H | H | H | 6-Hydroxyflavanone |
| H | H | OCH₃ | H | H | H | H | 6-Methoxyflavanone |
| H | H | H | OH | H | H | H | 7-Hydroxyflavanone |
| H | H | H | H | OH | H | H | 2′-Hydroxyflavanone |
| H | H | H | H | H | H | OH | 4′-Hydroxyflavanone |
| H | H | H | H | H | H | OCH3 | 4′-Methoxyflavanone |
| H | OH | H | OH | H | H | H | Pinocembrin |
| H | OH | H | OCH₃ | H | H | H | Pinocembrin-7-methylether |
| H | OH | H | OH | H | H | OH | Naringenin |
| H | OH | H | OH | H | H | OCH3 | Isosakuranetin |
| H | OH | H | OCH3 | H | H | OH | Sakuranetin |
| H | OH | H | Gla | H | H | OH | Naringenin-7-glucoside |
| H | OH | H | Rh-Glb | H | H | OH | Naringin |
| H | OH | H | OH | H | OH | OH | Eriodictyol |
| H | OH | H | OH | H | OCH₃ | OH | Homoeriodictyol |
| H | OH | H | OH | H | OH | OCH₃ | Hesperetin |
| OH | OH | H | OH | H | OH | OH | Taxifolin |

In one embodiment of the fourth aspect, the flavanone is dihydroxyflavanone and/or a (2S)-flavan-4-one, or a functional derivative thereof.

In one embodiment of the fourth aspect, the flavanone is (2S)-5,7-dihydroxy-2-phenyl-2,3-dihydrochromen-4-one, or a functional derivative thereof.

In one embodiment of the fourth aspect, the flavonoid is of the type naturally synthesized in a plant cell, although is not necessarily obtained from a plant cell for use in the method.

In one embodiment of the fourth aspect, use of the flavonoid in a sheep model of lung disease results in an improvement in any one or mode of lung function, presence of neutrophils and/or inflammatory cells in a lung lavage fluid, histologically assessed inflammation and/or fibrosis.

In one embodiment of the fourth aspect, the sheep model of lung disease relies on bleomycin-induced lung damage.

### BRIEF DESCRIPTION OF THE FIGURES

FIG. 1 shows graphically the timing of administration of (i) bleomycin (to elicit damage and fibrosis) and (ii) pinocembrin (the bioactive test compound) in the sheep study of Examples 1 and 2. Also shown is the timing of tissue sampling and performance of lung function tests.

FIG. 2 is a photograph of a sheep lung showing the three segments of the organ as treated in the study detailed in Examples 1 and 2. The right medial segment (designated “RM Sal” in following figures) was treated with saline only and represents a heathy lung control segment. The right caudal segment (designated “RC BLM” in following figures) was treated with bleomycin and vehicle, to represent a damaged lung segment. The left caudal segment (designated “LC BLM+PIN” in following figures) was treated with bleomycin and the bioactive test compound pinocembrin and represents a damaged but treated lung segment.

FIG. 3 is a graph showing the weights for the three sheep subject of the study described in Example 1.

FIG. 4A is graph showing lung function for each of the three lung segments for each sheep at week 11 after the sheep had received 4 weekly doses of pinocembrin.

FIG. 4B shows the same data as for FIG. 4A, except with lung function results for the three sheep being averaged and having error bars shown.

FIG. 5 is graph showing lung function at week 11 after sheep had received 4 weekly doses of pinocembrin in the study described in Example 1, with data represented as the change from baseline for each of the three lung segments. The results of an unpaired and paired sample t-test performed on the data is shown.

FIG. 6A is a graph showing neutrophils (inflammatory cells) in lung lavage fluids taken at week 12 after sheep had received 4 weekly doses of pinocembrin in the study described in Example 1, with data represented as the average for all three sheep for each of the three lung segments. The results of a paired sample t-test performed on the data is shown.

FIG. 6B is a graph showing the sum of inflammatory cells in lung lavage fluids taken at week 12 after sheep had received 4 weekly doses of pinocembrin in the study described in Example 1, with data represented as the average for all three sheep for each of the three lung segments. The results of a paired sample t-test performed on the data is shown.

FIG. 7A is a graph showing the same data as for FIG. 6A, except with data from each of the three sheep shown separately.

FIG. 7B is a graph showing the same data as for FIG. 6B, except that data from each of the three sheep are shown separately.

FIG. 8A is a graph showing the inflammation score in histology testing for each of the three lung segments at cull (week 12). The scores for the three sheep have been averaged and error bars provided.

FIG. 8B is a graph showing the fibrosis score in histology testing for each of the three lung segments at cull (week 12). The scores for the three sheep have been averaged, and error bars provided. The results of a paired sample t-test performed on the data is shown.

FIG. 8C is a graph showing the overall pathology score determined from the data presented in FIG. 8A and FIG. 8B for each of the three lung segments at cull (week 12). The scores for the three sheep have been averaged, and error bars provided. The results of a paired sample t-test performed on the data is shown.

FIG. 9A is a graph showing the same data as for FIG. 8A, except that data from each of the three sheep are shown separately.

FIG. 9B is a graph showing the same data as for FIG. 8B, except that data from each of the three sheep are shown separately.

FIG. 9C is a graph showing the same data as for FIG. 8C, except that data from each of the three sheep are shown separately.

FIG. 10A is a graph showing the overall disease score, scores calculated from lung function, pathology scores and BAL cells, as assessed in weeks 11+12. Scores have been normalised so that the maximum disease score in the RC BLM (bleomycin-infused, vehicle-treated) lung segment=100. The scores are shown as averaged across the three sheep, and the results of a paired sample t-test shown.

FIG. 10B is a graph showing the same data as for FIG. 10A, except that data from each of the three sheep are shown separately.

FIG. 11 is a graph showing weights of each sheep taken at specified times throughout the trial period detailed in Example 2

FIG. 12. Shows a series of graphs measuring lung function in the differentially treated lung segments as assessed at week 11 of the study detailed in Example 2. The differentially treated lung segments were the right medial (RM) lung-segments which were left untreated for healthy lung controls (Control), the right caudal (RC) and the left caudal (LC) lung-segments which were either infused with bleomycin without drug treatment (BLM), or infused with bleomycin and received 4 weekly doses of GA172 (BLM+GA172). GA172 is the code used for pinocembrin in this study. Part A shows mean data for Cseg (n=10), which is a measure for how easy it is to inflate the lung segment. Part B shows individual sheep data. Part C shows percent change of Cseg at week 11 from baseline values taken at week 0 at the beginning of the study. Significance was determined using paired t-tests, *p<0.05, **p<0.01, ***p<0.001, n=10 sheep.

FIG. 13. Shows a series of graphs measuring parameters of neutrophils and inflammatory cells recovered from the bronchoalveolar lavage (BAL) fluid of the differentially treated lung-segments at week 12. The differentially treated lung segments were the right medial (RM) lung-segments which were left untreated for healthy lung controls (Control), the right caudal (RC) and the left caudal (LC) lung-segments which were either infused with bleomycin without drug treatment (BLM) or infused with bleomycin and received 4 weekly doses of GA172 (BLM+GA172). The left panels show neutrophil data, and the right panels show inflammatory cell data, which included the sum of the percentages of neutrophils, lymphocytes, and eosinophils. The top panels show mean data for ten sheep. The bottom panel shows individual sheep data. Significance was determined using paired t-tests, *p<0.05, **p<0.01, ***p<0.001, n=10 sheep. GA172 is the code used for pinocembrin in this study.

FIG. 14 showing a series of graphs summarizing data for immuno-stained CD8+ and CD4+ T cells in the lung parenchyma sampled from the differentially treated lung-segments at week 12. The differentially treated lung segments were the right medial (RM) lung-segments which were left untreated for healthy lung controls (Control), the right caudal (RC) and the left caudal (LC) lung-segments which were either infused with bleomycin without drug treatment (BLM) or infused with bleomycin and received 4 weekly doses of GA172 (BLM+GA172). The left panels show mean lung segment data and the right panels show individual sheep data. Significance was determined using paired t-tests, *p<0.05, **p<0.01, ***p<0.001, n=10 sheep.GA172 is the code used for pinocembrin in this study.

FIG. 15. shows histopathology scoring data as assessed on histological H+E stained sections sampled at post-mortem from the differentially treated lung-segments. The differentially treated lung segments were the right medial (RM) lung-segments which were left untreated for healthy lung controls (Control), the right caudal (RC) and the left caudal (LC) lung-segments which were either infused with bleomycin without drug treatment (BLM) or infused with bleomycin and received 4 weekly doses of GA172 (BLM+GA172). The top panels show mean scoring data for ten sheep. The bottom panels show individual sheep data. Significance was determined using paired t-tests, *p<0.05, **p<0.01, n=10 sheep. Scoring criteria is described in the Materials and Methods. GA172 is the code used for pinocembrin in this study.

FIG. 16 shows data for the hydroxyproline assay to determine collagen content after four weekly treatments with GA172. Panel A shows data for 10 sheep participating in the trial of Example 2. Panel B shows data from 13 sheep participating in the trials of both Example 1 and Example 2. For each sheep, the differentially treated lung segments were the right medial (RM) lung-segments which were left untreated for healthy lung controls (Control), the right caudal (RC) and the left caudal (LC) lung-segments which were either infused with bleomycin without drug treatment (BLM), or infused with bleomycin and received 4 weekly doses of GA172 (BLM+GA172). The left panel shows mean data for thirteen sheep. The right panel shows individual sheep data. Significance was determined using paired t-tests, **p<0.01. GA172 is the code used for pinocembrin in this study.

FIG. 17 shows data for Masson's Trichrome stained connective tissue after four weekly treatments with GA172. Masson's Trichrome stains most connective tissue, including collagen, blue. For each sheep, the differentially treated lung segments were the right medial (RM) lung-segments which were left untreated for healthy lung controls (Control), the right caudal (RC) and the left caudal (LC) lung-segments which were either infused with bleomycin without drug treatment (BLM), or infused with bleomycin and received 4 weekly doses of GA172 (BLM+GA172). The left panel show mean scoring data for ten sheep. The right panel show individual sheep data. The staining and scoring methods are described in the Materials and Methods. Significance was determined using paired t-tests, *p<0.05, ***p<0.001, n=10 sheep. GA172 is the code used for pinocembrin in this study.

FIG. 18 shows a table referred to as “Table 1” in the description. Table 1 summarizes individual sheep data for all parameters assessed in Example 2. GA172 is the code used for pinocembrin in this study.

### DETAILED DESCRIPTION OF THE INVENTION AND PREFERRED EMBODIMENTS THEREOF

Throughout the description and the claims of this specification the word “comprise” and variations of the word, such as “comprising” and “comprises” is not intended to exclude other additives, components, integers or steps.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

The present invention is predicated at least in part on the inventors' discovery that a prototypical plant flavonoid is able to provide benefit in the prophylaxis and/or treatment of a pathological injury-induced fibrosis or inflammation. The flavonoid may act on fibrosis which does or does not follows inflammation. The flavonoid may act on inflammation that does or does not lead to fibrosis. Accordingly, the flavonoid may function as an anti-inflammatory and/or an anti-fibrosis agent. These discoveries are founded on the experimental studies in the Examples herein showing that pinocembrin (being an exemplary flavonone) is able to significantly improve disease parameters in an accepted animal model for idiopathic pulmonary fibrosis. That discovery may be applied to other fibrotic conditions including infection-induced pulmonary fibrosis (including infections of the respiratory tract from a coronavirus such as SARS-CoV-2), radiation-induced pulmonary fibrosis, progressive massive fibrosis, cystic fibrosis), pancreatic fibrosis (including cystic fibrosis), retropertinoneal fibrosis, arterial fibrosis (including arterial stiffness), intestinal fibrosis (including Crohn's disease), joint fibrosis (including athrofibrosis of the knee, shoulder and other joints, adhesive capsulitis), manual/digital fibrosis (including Dupuytren's contracture), dermal fibrosis (including keloid, nephrogenic systemic fibrosis, scleroderma), penis (including Peyronie's disease), lymph node fibrosis (including mediastinal fibrosis) and myocardial fibrosis (including interstitial fibrosis and replacement fibrosis).

In the context of the present invention, the term “fibrosis” refers to the formation or development of excess fibrous connective tissue in an organ or tissue as a result of injury or inflammation of a part, or of interference with its blood supply. It may be a consequence of the normal healing response leading to a scar, an abnormal, reactive process, with or without a known or understood causation.

A bonus effect of the studies on fibrosis is the finding that a flavonoid is useful in the treatment and/or prophylaxis of inflammation. Applicant experimentally investigated inflammatory markers that arose as a result of the stimulus arising from the bleomycin-induced injury that is essential to the animal model used for fibrosis used by the inventors.

In the context of the present invention, the term inflammation includes activation of the mammalian immune response after exposure to a stimulus such as an infection, an irritant, an allergen or to cell damage. Inflammation may be considered as a type of innate immunity, as compared with adaptive immunity which is specific response to a certain pathogenic agent.

Inflammation may be considered acute or chronic, the former generally mediated by granulocytes, and the latter by mononuclear cells including monocytes and lymphocytes.

Acute inflammation may arise as an initial protective response of the body against an injurious or stimulus by maintaining tissue integrity and effecting tissue repair. Alternatively, the stimulus may be an allergic stimulus.

Acute inflammation may be instigated by cells including resident macrophages, dendritic cells, histiocytes, Kupffer cells, mastocytes, vascular endothelial cells, and vascular smooth muscle cells. Upon stimulus, these cells are activated releasing inflammatory mediating and sensitizing molecules for example pro-inflammatory cytokines, pro-inflammatory prostaglandins, leukotrienes, histamine, serotonin, neutral proteases, bradykinin and nitric oxide. These molecules modulate biological pathways reliant on cellular and acellular agents in the local vasculature, immune system, and the affected tissue site to propagate and amplify the inflammatory response.

Acute inflammatory response, typically characterized by vasodilatation increasing blood flow into the tissue thereby causing erythema which may extend beyond the site, an increase in blood vessel permeability causing edema. The response may alter the excitability of certain sensory neurons causing hypersensitivity and pain. A further effect may include release of inflammation-inducing molecules such as neuropeptides including substance P, calcitonin gene-related peptide (CGRP), prostaglandins, and amino acids like glutamate. A further component of an inflammatory may be an increase in the migration of leukocytes, mainly granulocytes, from the blood vessels into the tissue. An acute inflammatory response typically ceases when the inflammatory stimulus is removed.

An extended stimulus may lead to a chronic inflammatory response resulting in a progressive shift in cell types present in the affected tissue. Chronic inflammation may be considered as the contemporaneous destruction and healing of tissue, with the ultimate outcome being deleterious (typically tissue injury). Chronic inflammation is involved in a range of otherwise unrelated conditions including cardiovascular disease, cancer, allergies, obesity, diabetes, digestive system diseases, degenerative diseases, auto-immune disorders, and neurological disease.

Attempts to treat or prevent chronic inflammation have had limited success, possibly due to the complex etiology of chronic inflammation and the many participating inflammation mediating and sensitizing agents. The NSAID class of drugs may block endogenous anti-inflammatory responses, which in some instances may prolong or exacerbate chronic inflammation.

In the analysis of tissues obtained from the model animals, pinocembrin was found to exert a significant effect on the inflammatory response generated by the bleomycin-induced injury (which is required in the model for idiopathic pulmonary fibrosis) in the animals' lung tissue. Accordingly, it is proposed that flavonoids may be useful in the treatment or prevention of a range of inflammatory conditions of the respiratory system including the lungs, and also the airways.

For example, asthma and COPD are diseases of high global prevalence having an inflammatory component which cause significant morbidity and mortality. Both diseases have characteristic symptoms and functional abnormalities, with airway obstruction being the main feature.

Airway obstruction in asthma is reversible while for COPD abnormal expiratory flow does not markedly changed over extended periods. The inflammation in these diseases may be triggered by environmental allergens, occupational sensitizing agents, or viral respiratory infections. In COPD, any of the myriad of agents in cigarette smoke may trigger the inflammatory response seen.

In the context of the present invention, the term “flavonoid” is intended to include flavanols, flavones, and flavanones. In some embodiments of the composition the flavonoid is a flavanone, and in some embodiments a chiral flavanone existing as optical isomers, and in which case the flavanone may be either the D-form or the S-form. In some embodiments of the compositions the S-isomer is used in the present compositions.

In some embodiments, the flavonoid is the flavanone pinocembrin. Advantageously, pinocembrin is a naturally occurring compounding having a known safety profile. Moreover, the compound is not controlled to the extent that a medical practitioner must prescribe the compound.

Many health consumers highly prefer to take a natural substance. In the present case, pinocembrin may be freely taken prophylactically (to prevent pulmonary fibrosis arising from a respiratory infection that may be contracted in the future, for example) without fear of significant adverse effects. Thus, a flavonoid compound may be taken as a general means for addressing any pulmonary issues that may be experienced at a later date.

In addition or alternatively, the flavonoid compound may be administered after a disease process has commenced, and in which case given the general safety of many plant derived compounds the compound may be freely taken on its own or in combination with other treatments (pharmaceutical or otherwise).

In a method of the present invention, a flavonoid is administered to a subject. The terms “subject” and “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The subject is one in need of prevention or treatment of a fibrotic or inflammatory condition, which refers to a subject who suffers from (or will possibly suffer from in the future) a disease, disorder, condition, or pathological process.

The terms “treat”, “treating”, “prevent”, and “preventing”, includes abrogating, substantially inhibiting, slowing or reversing the progression of a disease, condition or disorder, substantially ameliorating clinical or symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

According the present methods, the flavonoid is administered to the subject in an “effective amount”. This is to be taken to include a therapeutically effective amount, having regard to the fibrotic or inflammatory condition concerned and the characteristics of the subject. According to some embodiments, the effective amount of the flavonoid compound of the pharmaceutical composition is of an amount from about 0.000001 mg/kg body weight to about 100 mg/kg body weight. According to another embodiment, the effective amount of the flavonoid compound of the pharmaceutical composition is of an amount from about 0.00001 mg/kg body weight to about 100 mg/kg body weight. According to another embodiment, the effective amount of the flavonoid compound of the pharmaceutical composition is of an amount from about 0.0001 mg/kg body weight to about 100 mg/kg body weight. According to another embodiment, the effective amount of the flavonoid compound of the pharmaceutical composition is of an amount from about 0.001 mg/kg body weight to about 10 mg/kg body weight. According to another embodiment, the effective amount of the flavonoid compound is of an amount from about 0.01 mg/kg body weight to about 10 mg/kg body weight. According to another embodiment, the effective amount of the flavonoid compound of the pharmaceutical composition is of an amount from about 0.1 mg/kg (or 100 μg/kg) body weight to about 10 mg/kg body weight. According to another embodiment, the effective amount of the flavonoid compound of the pharmaceutical composition is of an amount from about 1 mg/kg body weight to about 10 mg/kg body weight. According to another embodiment, the effective amount of the flavonoid compound of the pharmaceutical composition is of an amount from about 10 mg/kg body weight to about 100 mg/kg body weight. According to another embodiment, the effective amount of the flavonoid compound of the pharmaceutical composition is of an amount from about 2 mg/kg body weight to about 10 mg/kg body weight. According to another embodiment, the effective amount of the flavonoid compound of the pharmaceutical composition is of an amount from about 3 mg/kg body weight to about 10 mg/kg body weight. According to another embodiment, the effective amount of the flavonoid compound of the pharmaceutical composition is of an amount from about 4 mg/kg body weight to about 10 mg/kg body weight. According to another embodiment, the effective amount of the flavonoid compound of the pharmaceutical composition is of an amount from about 5 mg/kg body weight to about 10 mg/kg body weight. According to another embodiment, the effective amount of the flavonoid compound of the pharmaceutical composition is of an amount from about 20, 30, 40, 50, or 60 mg/kg body weight to about 100 mg/kg body weight, including. According to another embodiment, the effective amount of the flavonoid compound of the pharmaceutical composition is of an amount from about 70 mg/kg body weight to about 100 mg/kg body weight. According to another embodiment, the effective amount of the flavonoid compound of the pharmaceutical composition is of an amount from about 80 mg/kg body weight to about 100 mg/kg body weight. According to another embodiment, the effective amount of the flavonoid compound of the pharmaceutical composition is of an amount from about 90 mg/kg body weight to about 100 mg/kg body weight. According to another embodiment, the effective amount of the flavonoid compound of the pharmaceutical composition is of an amount from about 0.000001 mg/kg body weight to about 90 mg/kg body weight. According to another embodiment, the effective amount of the flavonoid compound of the pharmaceutical composition is of an amount from about 0.000001 mg/kg body weight to about 80 mg/kg body weight. According to another embodiment, the effective amount of the flavonoid compound of the pharmaceutical composition is of an amount from about 0.000001 mg/kg body weight to about 70 mg/kg body weight. According to another embodiment, the effective amount of the flavonoid compound of the pharmaceutical composition is of an amount from about 0.000001 mg/kg body weight to about 60 mg/kg body weight. According to another embodiment, the effective amount of the flavonoid compound of the pharmaceutical composition is of an amount from about 0.000001 mg/kg body weight to about 50 mg/kg body weight. According to another embodiment, the effective amount of the flavonoid compound of the pharmaceutical composition is of an amount from about 0.000001 mg/kg body weight to about 40 mg/kg body weight. According to another embodiment, the effective amount of the flavonoid compound is of an amount from about 0.000001 mg/kg body weight to about 30 mg/kg body weight. According to another embodiment, the effective amount of the flavonoid compound of the pharmaceutical composition is of an amount from about 0.000001 mg/kg body weight to about 20 mg/kg body weight. According to another embodiment, the effective amount of the flavonoid compound of the pharmaceutical composition is of an amount from about 0.000001 mg/kg body weight to about 10 mg/kg body weight. According to another embodiment, the effective amount of the flavonoid compound of the pharmaceutical composition is of an amount from about 0.000001 mg/kg body weight to about 1 mg/kg body weight. According to another embodiment, the effective amount of the flavonoid compound of the pharmaceutical composition is of an amount from about 0.000001 mg/kg body weight to about 0.1 mg/kg body weight. According to another embodiment, the effective amount of the flavonoid compound of the pharmaceutical composition is of an amount from about 0.000001 mg/kg body weight to about 0.1 mg/kg body weight. According to another embodiment, the effective amount of the flavonoid compound of the pharmaceutical composition is of an amount from about 0.000001 mg/kg body weight to about 0.01 mg/kg body weight. According to another embodiment, the effective amount of the flavonoid compound of the pharmaceutical composition is of an amount from about 0.000001 mg/kg body weight to about 0.001 mg/kg body weight. According to another embodiment, the effective amount of the flavonoid compound of the pharmaceutical composition is of an amount from about 0.000001 mg/kg body weight to about 0.0001 mg/kg body weight. According to another embodiment, the effective amount of the flavonoid compound of the pharmaceutical composition is of an amount from about 0.000001 mg/kg body weight to about 0.00001 mg/kg body weight.

According to some other embodiments, the therapeutic dose of the flavonoid compound of the pharmaceutical composition ranges from 1 μg/kg/day to 25 μg/kg/day. According to some other embodiments, the therapeutic dose of the flavonoid compound of the pharmaceutical composition ranges from 1 μg/kg/day to 2 μg/kg/day. According to some other embodiments, the therapeutic dose of the flavonoid compound of the pharmaceutical composition ranges from 2 μg/kg/day to 3 μg/kg/day. According to some other embodiments, the therapeutic dose of the flavonoid compound of the pharmaceutical composition ranges from 3 μg/kg/day to 4 μg/kg/day. According to some other embodiments, the therapeutic dose of the flavonoid compound of the pharmaceutical ranges from 4 μg/kg/day to 5 μg/kg/day. According to some other embodiments, the therapeutic dose of the flavonoid compound of the pharmaceutical composition ranges from 5 μg/kg/day to 6 μg/kg/day. According to some other embodiments, the therapeutic dose of the flavonoid compound of the pharmaceutical composition ranges from 6 μg/kg/day to 7 μg/kg/day. According to some other embodiments, the therapeutic dose of the flavonoid compound of the pharmaceutical composition ranges from 7 μg/kg/day to 8 μg/kg/day. According to some other embodiments, the therapeutic dose of the flavonoid compound of the pharmaceutical composition ranges from 8 μg/kg/day to 9 μg/kg/day. According to some other embodiments, the therapeutic dose of the flavonoid compound of the pharmaceutical composition ranges from 9 μg/kg/day to 10 μg/kg/day. According to some other embodiments, the therapeutic dose of the flavonoid compound of the pharmaceutical composition ranges from 1 μg/kg/day to 5 μg/kg/day. According to some other embodiments, the therapeutic dose of the flavonoid compound of the pharmaceutical composition ranges from 5 μg/kg/day to 10 μg/kg/day. According to some other embodiments, the therapeutic dose of the flavonoid compound of the pharmaceutical composition ranges from 10 μg/kg/day to 15 μg/kg/day. According to some other embodiments, the therapeutic dose of the flavonoid compound of the pharmaceutical composition ranges from 15 μg/kg/day to 20 μg/kg/day. According to some other embodiments, the therapeutic dose of the flavonoid compound of the pharmaceutical composition ranges from 25 μg/kg/day to 30 μg/kg/day. According to some other embodiments, the therapeutic dose of the flavonoid compound of the pharmaceutical composition ranges from 30 μg/kg/day to 35 μg/kg/day. According to some other embodiments, the therapeutic dose of the flavonoid compound of the pharmaceutical composition ranges from 35 μg/kg/day to 40 μg/kg/day. According to some other embodiments, the therapeutic dose of the flavonoid compound of the pharmaceutical composition ranges from 40 μg/kg/day to 45 μg/kg/day. According to some other embodiments, the therapeutic dose of the flavonoid compound of the pharmaceutical composition ranges from 45 μg/kg/day to 50 μg/kg/day. According to some other embodiments, the therapeutic dose of the flavonoid compound of the pharmaceutical composition ranges from 50 μg/kg/day to 55 μg/kg/day. According to some other embodiments, the therapeutic dose of the flavonoid compound of the pharmaceutical composition ranges from 55 μg/kg/day to 60 μg/kg/day. According to some other embodiments, the therapeutic dose of the flavonoid compound of the pharmaceutical composition ranges from 60 μg/kg/day to 65 μg/kg/day. According to some other embodiments, the therapeutic dose of the flavonoid compound of the pharmaceutical composition ranges from 65 μg/kg/day to 70 μg/kg/day. According to some other embodiments, the therapeutic dose of the flavonoid compound of the pharmaceutical composition ranges from 70 μg/kg/day to 75 μg/kg/day. According to some other embodiments, the therapeutic dose of the flavonoid compound of the pharmaceutical composition ranges from 80 μg/kg/day to 85 μg/kg/day. According to some other embodiments, the therapeutic dose of the flavonoid compound of the pharmaceutical composition ranges from 85 μg/kg/day to 90 μg/kg/day. According to some other embodiments, the therapeutic dose of the flavonoid compound of the pharmaceutical composition ranges from 90 μg/kg/day to 95 μg/kg/day. According to some other embodiments, the therapeutic dose of the flavonoid compound of the pharmaceutical composition ranges from 95 μg/kg/day to 100 μg/kg/day.

An effective amount of a flavonoid of the described invention includes, but is not limited to, an amount sufficient: (1) to remove, or decrease the size of, at least one fibrotic locus or (2) to reduce the rate of extracellular matrix, including collagen and fibronectin, deposition in the interstitia in the lungs of a pulmonary fibrosis patient, or (3) inflammation, including in the influx of inflammatory cells such as neutrophils to the affected tissue. The term also encompasses an amount sufficient to suppress or alleviate at least one symptom of a pulmonary fibrosis patient, wherein the symptom includes, but is not limited to, oxygen saturation, dyspnea (difficulty breathing), nonproductive cough (a sudden, noisy expulsion of air from the lungs that may be caused by irritation or inflammation and does not remove sputum from the respiratory tract, and crackles (crackling sound in lungs during inhalation, sometimes referred to as rales or crepitations). The term “effective amount” may also encompass an amount sufficient to prevent or at least partially reverse the coughing, wheezing or narrowing of an airway a seen in asthma and COPD. The term may also encompass an amount sufficient to prevent or at least partially reverse the coughing, wheezing or mucous production seen in acute or chronic bronchitis.

An effective amount of an active agent that can be employed according to the described invention generally ranges from generally about 0.001 mg/kg body weight to about 10 g/kg body weight. However, dosage levels are based on a variety of factors, including the type of injury, the age, weight, sex, medical condition of the patient, the severity of the condition, the route and frequency of administration, and the particular active agent employed. Thus the dosage regimen may vary widely, but can be determined routinely by a physician using standard methods, and having the benefit of the present specification.

Where the flavonoid is to be delivered to the lungs, inhalation (the act of drawing in a medication with the breath) or insufflation (the act of delivering air, a gas, or a powder under pressure to a cavity or chamber of the body, for example, nasal insufflation relates to the act of delivering air, a gas, or a powder under pressure through the nose) may be exploited as a route of administration.

The flavonoid compound may be delivered with the assistance of an inhalation device, which may be a machine/apparatus or component that produces small droplets or an aerosol from a liquid or dry powder aerosol formulation and is used for administration through the mouth in order to achieve pulmonary administration of a drug, e.g., in solution, powder, and the like. Examples of inhalation delivery device include, but are not limited to, a nebulizer, a metered-dose inhaler, and a dry powder inhaler (DPI).

The term “nebulizer” as used herein refers to a device used to administer liquid medication in the form of a mist inhaled into the lungs.

The term “metered-dose inhaler”, “MDI”, or “puffer” as used herein refers to a pressurized, hand-held device that uses propellants to deliver a specific amount of medicine (“metered dose”) to the lungs of a patient. The term “propellant” as used herein refers to a material that is used to expel a substance usually by gas pressure through a convergent, divergent nozzle. The pressure may be from a compressed gas, or a gas produced by a chemical reaction. The exhaust material may be a gas, liquid, plasma, or, before the chemical reaction, a solid, liquid or gel. Propellants used in pressurized metered dose inhalers are liquefied gases, traditionally chlorofluorocarbons (CFCs) and increasingly hydrofluoroalkanes (HFAs). Suitable propellants include, for example, a chlorofluorocarbon (CFC), such as trichlorofluoromethane (also referred to as propellant 11), dichlorodifluoromethane (also referred to as propellant 12), and 1,2-dichloro-1,1,2,2-tetrafluoroethane (also referred to as propellant 114), a hydrochlorofluorocarbon, a hydrofluorocarbon (HFC), such as 1,1,1,2-tetrafluoroethane (also referred to as propellant 134a, HFC-134a, or HFA-134a) and 1,1,1,2,3,3,3-heptafluoropropane (also referred to as propellant 227, HFC-227, or HFA-227), carbon dioxide, dimethyl ether, butane, propane, or mixtures thereof. In other embodiments, the propellant includes a chlorofluorocarbon, a hydrochlorofluorocarbon, a hydrofluorocarbon, or mixtures thereof. In other embodiments, a hydrofluorocarbon is used as the propellant. In other embodiments, HFC-227 and/or HFC-134a are used as the propellant.

The term “dry powder inhaler” or “DPI” as used herein refers to a device similar to a metered-dose inhaler, but where the drug is in powder form. The patient exhales out a full breath, places the lips around the mouthpiece, and then quickly breathes in the powder. Dry powder inhalers do not require the timing and coordination that are necessary with MDIs.

The term “particles” as used herein refers to refers to an extremely small constituent (e.g., nanoparticles, microparticles, or in some instances larger) in or on which is contained the composition as described herein.

It is proposed that for pulmonary applications, it may not be necessary to deliver the flavonoid directly to lung tissues and in some circumstances the compound may be administered orally, parenterally, rectally or by some other route of administration. Furthermore, for non-pulmonary applications indications the flavonoid will be generally administered via non-pulmonary routes.

In that regard, the present methods may require the administration of a pharmaceutical composition or a single unit dosage form comprising a flavonoid of the invention, or a pharmaceutically acceptable salt, hydrate or stereoisomer thereof, that are also encompassed by the invention. Individual dosage forms of the invention may be suitable for oral, mucosal (including sublingual, buccal, rectal, nasal, or vaginal), parenteral (including subcutaneous, intramuscular, bolus injection, intra-arterial, or intravenous), transdermal, or topical administration. Pharmaceutical compositions and dosage forms of the invention typically also comprise one or more pharmaceutically acceptable excipients. Sterile dosage forms are also contemplated.

A pharmaceutical composition encompassed by this embodiment includes a flavonoid of the invention, or a pharmaceutically acceptable salt, hydrate or stereoisomer thereof, and at least one additional therapeutic agent such as a prior art composition for the treatment of the relevant fibrotic or inflammatory condition. The composition, shape, and type of dosage forms will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein.

Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses use of anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. In effect, water and heat accelerate the decomposition of some compounds.

Thus, the effect of water on a formulation can be of significance since moisture and/or humidity are commonly encountered during manufacture, handling, packaging, storage, shipment, and use of formulations.

Anhydrous pharmaceutical compositions and dosage forms for use with the invention can be prepared using anhydrous or low moisture containing ingredients and low moisture or low humidity conditions.

An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise flavonoids of the invention, or a pharmaceutically acceptable salt, hydrate, or stereoisomers thereof comprise 0.1 mg to 1500 mg per unit to provide doses of about 0.01 to 200 mg/kg per day.

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), macrocrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVTCEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, co oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The flavonoid used in the methods of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g, adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Parenteral dosage forms can be administered to patients by various routes including, but not limited to, subcutaneous, intravenous (including bolus injection), intramuscular, and intraarterial. Because their administration typically bypasses patients' natural defences against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl rnyristate, and benzyl benzoate.

Compounds that increase the solubility of one or more of the active ingredients disclosed herein can also be incorporated into the parenteral dosage forms of the invention.

Transdermal dosage forms may be used. Such forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend oα the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrotidones such as polyvinylpyrrolidone; ollidon grades (Povidone, Polyvidone); urea; and various water-soluble or insoluble sugar esters such as Tween 80 (polysoibate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

Where the fibrotic or inflammatory condition has dermal or subdermal involvement, topical dosage forms may be used. Such forms include, but are not limited to, creams, lotions, ointments, gels, solutions, emulsions, suspensions, or other forms known to one of skill in the art.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrafuryl; alkyl sulfoxides such as dimethyl sulfoxide; dimethyl formamide; polyethylene glycol; pyrrolidones such as polyvinylpyrrolidone; kollidon grades (Povidone, Polyvidone); urea; and various water-soluble or insoluble sugar esters such as Tween 80 (polysorbate 80) and Span 60 (sorbitan monostearate).

Mucosal dosage forms may be used which include, but are not limited to, ophthalmic solutions, sprays and aerosols, or other forms known to one of skill in the art. Dosage forms suitable for treating mucosal tissues within the oral cavity can be formulated as mouthwashes or as oral gels. In one embodiment, the aerosol comprises a carrier. In another embodiment, the aerosol is carrier free.

The flavonoids of the invention may also be administered directly to the lung by inhalation. For administration by inhalation, a flavonoid can be conveniently delivered to the lung by a number of different devices.

A flavonoid can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver flavonoids. Certain organic solvents such as dimethylsulfoxide can also be employed, although usually at the cost of greater toxicity. A flavonoid can also be delivered in a controlled release system. In one embodiment, a pump can be used. In another embodiment, polymeric materials can be used. In yet another embodiment, a controlled-release system can be placed in proximity of the target of the compounds of the invention, e.g., the lung, thus requiring only a fraction of the systemic dose.

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl rnyristate, isopropyl palmitate, mineral oil, and mixtures thereof, which are non-toxic and pharmaceutically acceptable.

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

In some embodiments of the present invention a flavonoid is formulated or otherwise administered in combination with an amino acid. It is proposed that the flavonoid and amino acid act synergistically so as to treat of prevent and inflammatory condition, and particularly an inflammatory condition of the airways such as asthma and acute respiratory distress syndrome (such as found in coronavirus infection, COVID-19). Particularly efficacious amino acids include L-glycine and L-tyrosine. Gut bacterial metabolites such as p-cresol-sulfate may be combined with a flavonoid, and a third active may be combined such as an amino acid.

The flavonoids may be incorporated into nutritional products including, but not limited to food compositions, over the counter, and dietary supplements. The flavonoids may be added to various foods so as to be consumed simultaneously. As a food additive, the flavonoids of the invention may be used in the same manner as conventional food additives, and thus, only needs to be mixed with other components to enhance the taste.

It will be recognized that dietary supplements may not use the same formulation ingredients or have the same sterile and other drug regulatory agency requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements may take the form of tablets or capsules prepared by conventional means and optionally including other dietary supplements including vitamins, minerals, other herbal supplements, binding agents, fillers, lubricants, disintegrants, or wetting agents, as those discussed above, The tablets may be coated by methods well-known in the art. In a preferred embodiment, the dietary supplement may take the form of a capsule or powder to be dissolved in a liquid for oral consumption.

The amount of flavonoid in a beverage or incorporated into a food product will depend on the kind of beverage, food and the desired effect. In general, a single serving comprises an amount of about 0.1% to about 50%, preferably of about 0.5% to about 20% of the food composition. More preferably a food product comprises flavonoids in an amount of about 1% to about 10% by weight of the food composition. Examples of food include, but are not limited to, confectionery such as sweets (candies, jellies, jams, etc.), gums, bean pastes, baked confectioneries or molded confectioneries (cookies, biscuits, etc.), steamed confectioneries, cacao or cacao products (chocolates and cocoa), frozen confectioneries (ice cream, ices, etc.), beverages (fruit juice, soft drinks, carbonated beverages), health drinks, health bars, and tea (green tea, black tea, etc.).

Pinocembrin is a preferred flavonoid according to the present invention. There are three main methods of production of pinocembrin. Extraction of pinocembrin may use as a starting material, a plant material, honey or propolis, and fungi for example.

For reason of cost, efficiency or consumer acceptance, the compound may be preferably extracted from a natural source. The compound is present in a wide variety of plants but is more prevalent in some families. It does not uniformly occur in a particular part of the plant, but each family tends to concentrate it in the same area. It is thought to perform a protective function for the plant in case of pathogen attack. The majority of plants appear to contain (S)-pinocembrin, but some contain the (R)-enantiomer or racemic material.

Many Eucalyptus species contain pinocembrin, and some to very high levels. For example Eucalyptus torelliana may express the compound to a level of 3.7% in fruit resin. Lower levels are found in leaf material, although nevertheless sufficient to provide for practical and economical extraction.

Some of the highest yields of pinocembrin come from Alpinia species, in fact Alpinia katsumadai appears to be a prime commercial source. The yields reported for this species range from 613 mg/kg to 2490 mg/kg from the seeds. 32000 mg/kg has been isolated from the rhizomes of Alpinia officinarium.

The leaves of Glycyrrhiza glabra (liquorice) are reported to have a particularly high level of pinocembrin, up to 24100 mg/kg.

Pinocembrin has been detected in monofloral honey of Leptospermum polygalifolium and Leptospermum scoparium. This indicates that the nectar of these plants contain pinocembrin, and at a level of 60 to 260 mg/kg.

Pinocembrin has been isolated from the flowers of Syzygium jambos and the leaves and fruit of S. samarangense. The content in the fruit is not particularly high at 2.2 mg/kg, although usable.

Preferably, pinocembrin is isolated from a plant source without using chromatography. If chromatography must be used, it should be as late as possible in the process to minimise the complexity of the extract and the volumes of solvent required.

A crude mixture of only 3 flavanoids was isolated from Eucalyptus sieberi by the following steps. Extraction with methanol at room temperature followed by partial concentration, followed by pouring into water and filtering off the precipitate. Repeated re-dissolution of the precipitate in methanol and re-precipitation with water until no flavanoids remained in the precipitate. Concentration of the combined aqueous methanol solutions and extraction of chlorophyll and wax with petroleum spirit. Partial concentration of the petroleum spirit and liquid-liquid extraction with ether for several days. Partial concentration and precipitation in the cold followed by separation by chromatography.

An alternative process to the concentration of large volumes of aqueous methanol would be to pass it through a macroporous resin such as XAD, carry out gradient elution and collect and concentrate the target fractions. Chromatography is nevertheless required.

Crude extracts containing flavanoids have been obtained from dry leaves by room temperature extraction, and by soxhlet extraction with n-hexane60 or methanol. Soxhlet extraction uses less solvent than cold extraction and indicates that pinocembrin can survive up to 68° C. for extended periods. Extraction with methanol was investigated using soxhlet extraction (64.7° C., 32 h), ultrasonic assisted extraction (ultrasound, 40° C. 30 min thrice) and accelerated solvent extraction at 60° C. (100 bar, 20 min, two cycles), 80° C. (100 bar, 20 min, two cycles), and 100° C. (100 bar, 20 min, two cycles) which gave 3.2 g, 2.6 g, 3.3 g, 3.6 g and 3.5 g of extract respectively.

Pinocembrin for the present compositions may be obtained by fermentation methods in Escherichia coli, Saccharomyces cerevisiae and Streptomyces venezuelae. The first two appear to produce (S)-pinocembrin but S. venezuelae produces a racemate.

Cell culture is proposed as a means for production of plant-derived metabolites (including pinocembrin) as it has the potential to accumulate higher quantities than an intact plant. Members of the family Zingiberaceae produce significant quantities of pinocembrin. Up to 9.2 g/kg has been reported for Boesenbergia rotunda, a member of this family. Cell suspension cultures have been established using a meristem-derived callus using a medium of naphthyl acetic acid and 2,4-dichlorophenoxyacetic acid. Inoculation at 1.0 mL of settled cell volume led to the maximum accumulation of pinocembrin at 8.6 mg/kg of dry weight.

There are a number of chemical syntheses of pinocembrin reported in the literature. For example, pinocembrin can be biosynthesised from L-phenylalanine. Four catalytic steps are required for this conversion. First, L-phenylalanine is converted to cinnamic acid by phenylalanine ammonia lyase (PAL). Cinnamic acid is then converted into the corresponding coenzyme A (CoA) ester by 4-coumarate:CoA ligase (4CL). Three molecules of malonyl-CoA are then condensed stepwise with one molecule of the cinnamyl-CoA ester to give (2S)-pinocembrin chalcone, catalysed by chalcone synthase (CHS). Finally chalcone isomerase (CHI) converts chalcone to (2S)-pinocembrin.

In some embodiments of the composition the flavanone is pinocembrin and preferably (S)-pinocembrin as shown below.

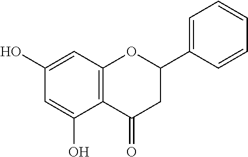 [SMILES: O=C1CC(c2ccccc2)Oc2cc(O)cc(O)c21]

Other dihydroxyflavanones may be used in place of pinocembrin for example, 4′,7-Dihydroxyflavanone (liquiritigenin) may be used.

In some embodiments a monohydroxylflavanone such as pinostrobin (being a (2S)-flavanone substituted by a hydroxyl group at position 5 and a methoxy group at position 7.

Other potentially useful compounds include the flavanones chrysin, galangin and pinobanksin.

### Example 1: Demonstration of Efficacy of 5,7-Dihydroxy-2-Phenyl-2,3-Dihydro-4H-Chromen-4-One (Pinocembrin) in the Treatment of Inflammation and Fibrosis in a Sheep Pulmonary Model

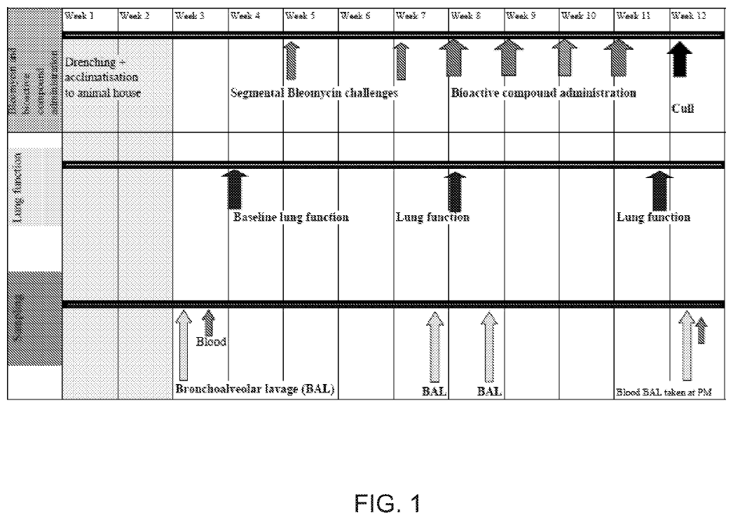

In animal models, bleomycin is the most widely used agent to characterise pulmonary fibrosis. In the sheep model, intratracheal administrations of two doses of bleomycin is used to induce fibrosis in the lung parenchyma. The overall study protocol is shown at FIG. 1.

Experimental Animals

For this example, three female merino sheep aged between 9 months and 1 year were utilised. Animals were housed indoors and received anthelminthic to treat for any existing parasitic disease. The sheep were judged to be free from significant pulmonary disease on the basis of clinical examination before the commencement of experiments and on inspection of gross pathology at autopsy. The Animal Experimentation Ethics Committee of the University of Melbourne, which adheres to the Australian Code of Practice for the care and use of laboratory animals for scientific purposes, approved all experimental procedures outlined below.

Bleomycin Administration and Treatment Protocols

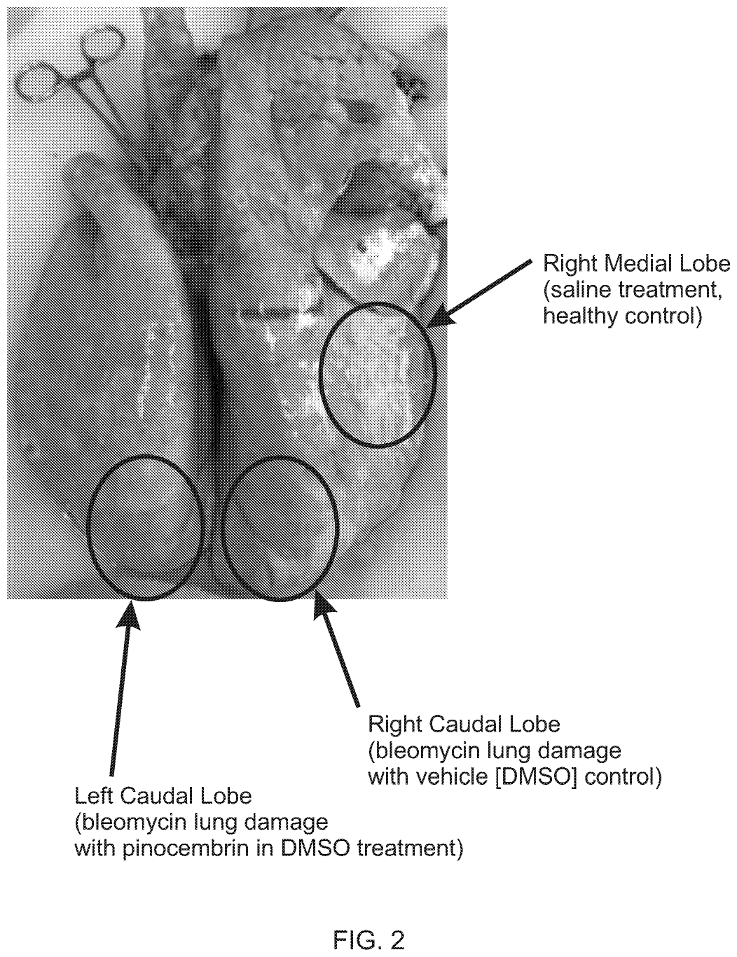

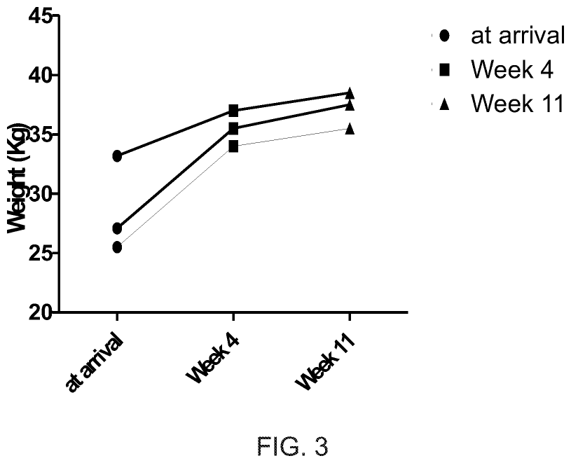

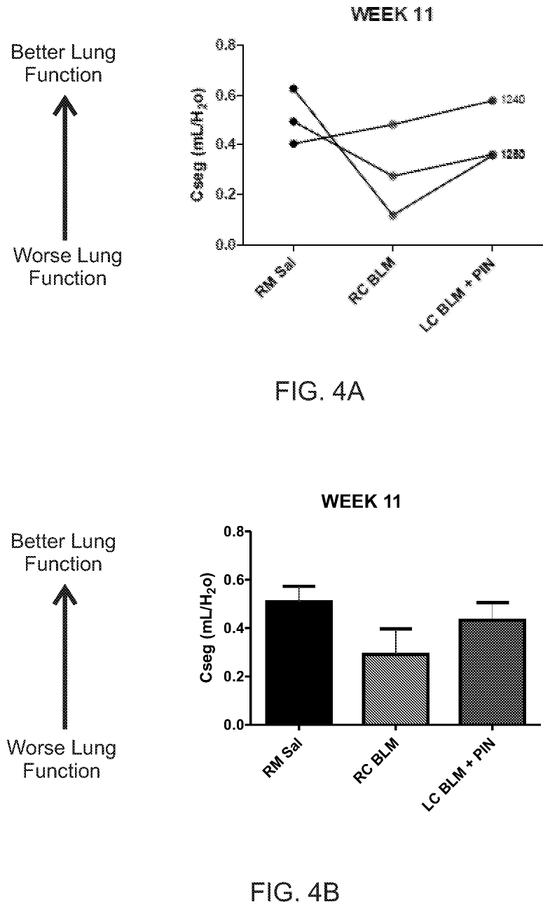

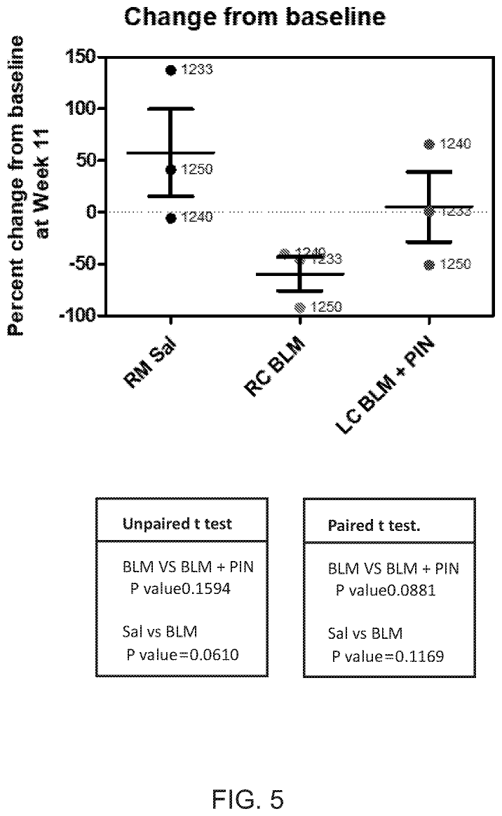

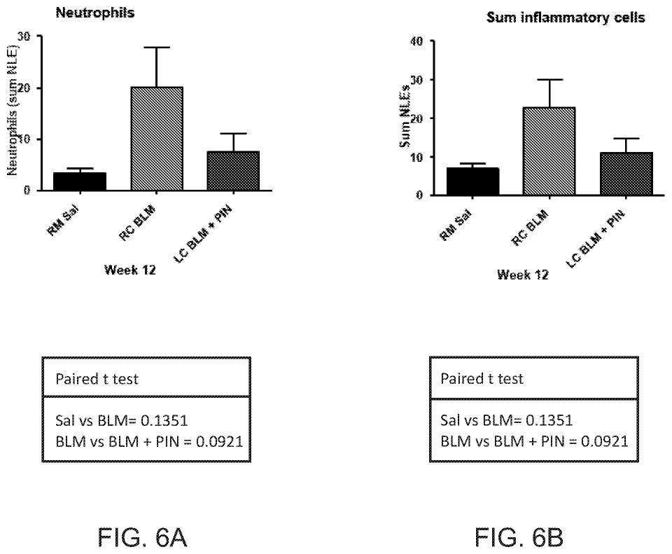

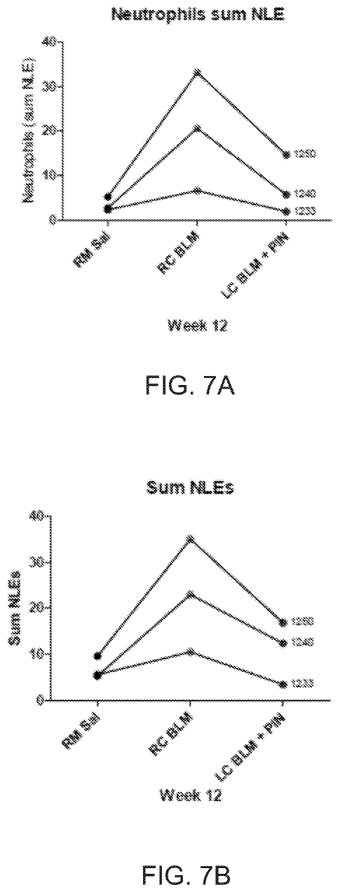

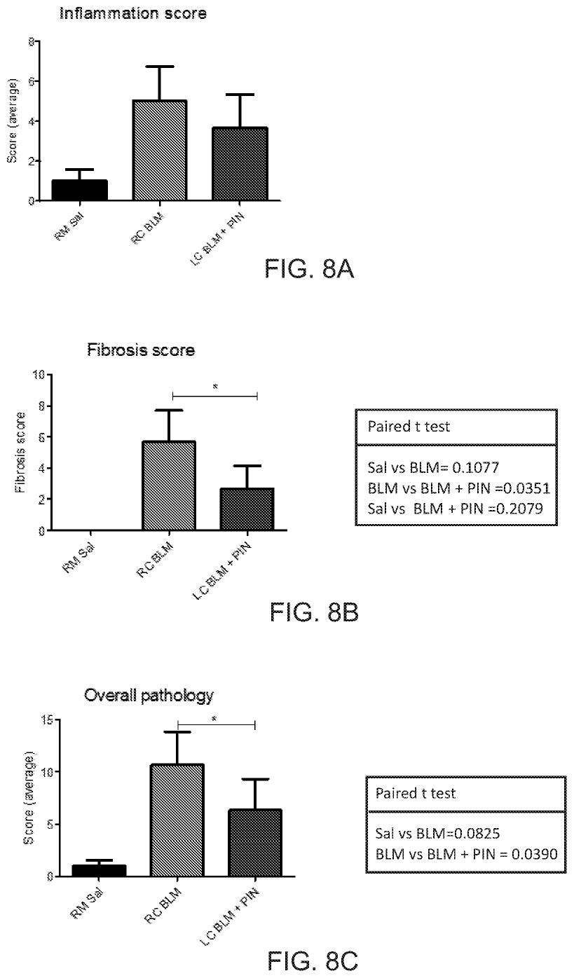

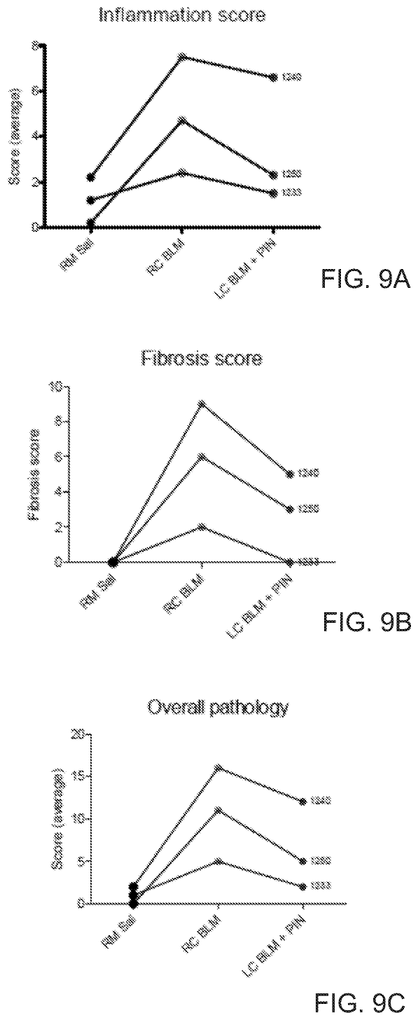

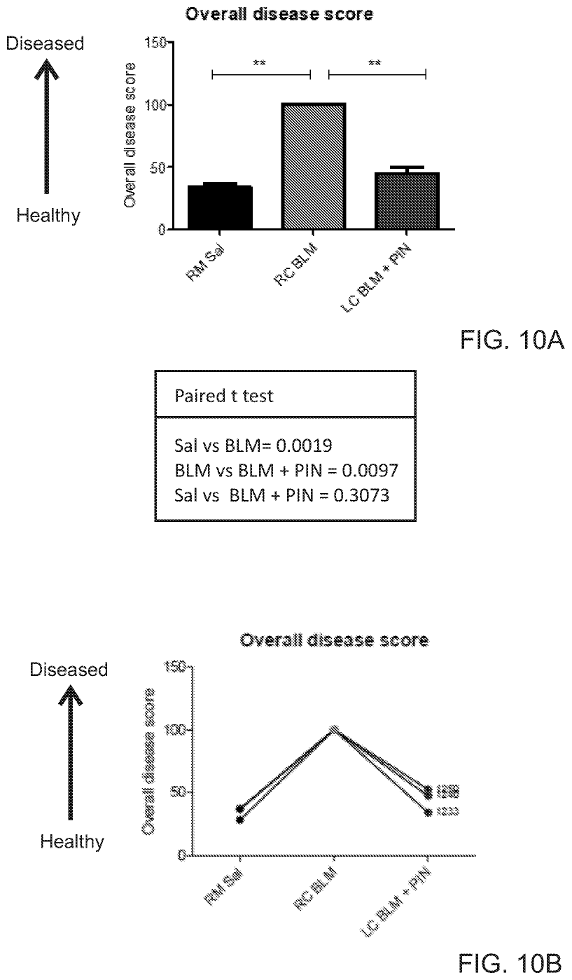

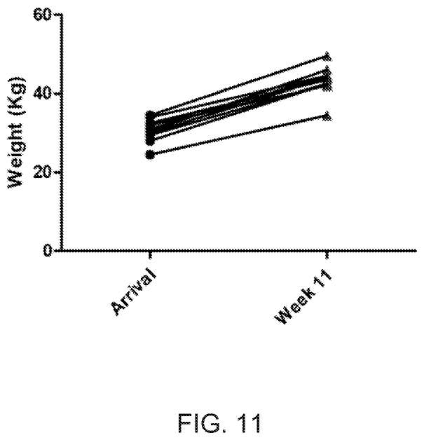

Fibrosis was induced in living sheep within the left caudal lobe of the lung of all animals, as indicated in FIG. 2, using pharmaceutical grade bleomycin sulphate (Hospira, Melbourne, Australia). Bleomycin sulphate was made up at a concentration of 0.6 U bleomycin/mL saline and administered to the left and right caudal lobes at a rate of 3 U per segment to cause injury to the tissue and trigger fibrosis. For the left caudal lobe, 7 mg of pinocembrin in 10% DMSO was administered to test the efficacy of pinocembrin in the treatment or prevention of bleomycin-induced fibrosis. For the right caudal lobe injury was induced by bleomycin, and DMSO alone administered such that any differential effects between the left and right caudal lobes could be attributed to the pinocembrin.

A saline solution was administered to the right medial lobe, as a sham treatment.

Each of the bleomycin, bleomycin/pinocembrin, and saline compositions was administered via the biopsy port of a fibre-optic bronchoscope to the appropriate lung segments as a 5 ml bolus.

Timing of the administration of the various compositions is summarised at FIG. 1, with all three sheep euthanized at week 12.

Necropsy and Tissue Sampling

The sheep were euthanized by an intravenous overdose of barbiturate (Lethabarb, Veterinary Clinic, University of Melbourne, Werribee, Australia) at week 12 as outlined in FIG. 1 for tissue collection and analysis.

Following euthanasia, the lungs were removed and targeted lung segments identified and carefully dissected free from surrounding tissue. Individual segments were then inflated with a 1:1 mixture of OCT and sterile PBS solution. This inflation procedure maintains lung segment tissues in an inflated state prior to either fixation in formalin, or freezing for cryo-sectioning.

2 mm thick transverse slices were taken and each treated segment was fixed in 10% neutral-buffered formalin and processed in paraffin for histopathology assessment. Remaining lung slices were embedded in OCT and frozen in cryo-moulds on aluminium boats floating on liquid nitrogen. These were kept at −80° C. for cryo-sectioning and immmunohistology.

Analysis of Segmental Lung Function

Segmental compliance (Cseg) was assessed using pressure responses to flow in the different segments as indicated FIG. 2 in awake, consciously breathing sheep using a wedged-bronchoscope technique. Briefly, a custom-built Segmental Lung Airway Monitoring (SLAM) System was used to monitor the segmental flow and pressure. After first determining the bronchoscope resistance to the set flow, the bronchoscope was wedged into an airway in the lung segment of interest and a constant flow (6 mL/s) of 5% C02 in air was passed through the working channel of the bronchoscope.

Segmental lung compliance was calculated. Briefly, after the bronchoscope was wedged into the specific region of the lung, pressure was allowed to reach a steady state. After approximately 5 s at steady state, airflow was interrupted turning off the airflow supply. Segmental compliance was then calculated from the pressure-flow decay curve generated from this procedure. The process was repeated 3 times for each segment and expressed as an average value for Cseg. The pressure was recorded by a PM-1000 Transducer Amplifiers (CWE Inc., Admore, USA) and flow was recorded using a mass flow meter (824-S, Sierra Instruments, Monterey, USA). Data were acquired with Data Acquisition Card (PCI-6233; National Instruments Corp., Austin, USA) and was analysed with the SLAM system (Latitude E6520, Dell Computer Corporation, Texas USA and LabVIEW, National Instruments Corp., Austin, USA). All resistance measurements were corrected for the resistance of flow through the working channel of the bronchoscope.

Histology

Paraffin-embedded tissue sections (5 μm) were stained with haematoxylin and eosin Y (H&E) for general histology and the assessment of pathological changes and Masson's trichrome staining was used to identify changes to collagen content within the lung parenchyma. Fibrotic lung injury was assessed morphologically by semi-quantitative and quantitative parameters as follows:

(i) Semi-Quantitative Morphological Index (SMI)

Histopathology of lung parenchyma was assessed by an experienced pathologist blinded to the treatment groups using a semi-quantitative scoring system. Briefly, the criteria used gives score indices separately for both inflammation and fibrosis pathology, and these score indices added together give an ‘overall pathology score’.

(ii) Quantitative Image Analysis (QIA)

a) Fibrosis fraction: The degree of fibrosis, or collagen content, was quantified to give an indication of changes for overall collagen content within the parenchymal tissue. Briefly, Masson's trichrome stained slides were scanned into a digital format using Mirax slide scanner (Carl Zeiss Micro-Imaging, Jena, Germany). Ten consecutive, non-overlapping fields were selected for analysis, which lacked obvious airways and/or blood vessels. Each field was analysed using Image Pro plus (Version 6.3 for Windows, Media Cybernetics, Bethesda, Md., USA), using the ‘Colour selector’ tool to measure the area blue stained tissue (collagen) within the each field. The fraction of blue stained collagen areas for each of the ten fields was averaged for each slide. The area of the fraction of fibrosis is expressed as a percentage of the total field area.

b) Morphometrics: Paraffin-embedded sections of lung tissue were stained with H&E for morphometric assessment. Digital images of lung parenchyma from control- and bleomycin-treated lung segments were imported into Image Pro Plus software for analysis. Measurements were made by superimposing custom-designed test grids over the lung parenchyma, which were generated using Image Pro. Tissue and airspace fractions were determined within parenchymal tissue by point-counting methods. Analyses were performed from a total of 15 fields at a final magnification of 200×.

Immunohistochemistry

Immunohistochemistry was performed on frozen tissue sections. Sections were fixed with 100% cold ethanol for 10 min and were simultaneously blocked for endogenous peroxidase with 3% H₂O₂(Univar, Knoxville, Vic, Australia). Sections were then pre-blocked using blocking solution for 30 min (1% bovine serum albumin, 5% normal sheep serum in PBS). After blocking, sections were incubated with the primary antibodies for CD4 and CD8 positive inflammatory cells (each being mouse antibodies obtained from AbD Serotech, Raleigh, USA).

After washing, sections were incubated for with appropriate secondary antibodies (rabbit anti-mouse Ig/HRP; AbD Serotech, Raleigh, USA) for 1 h. Sections were then washed and a peroxidase-based detection system was used for visualization. Specificity was determined by omission of the primary antibody on secondary controls, and biologically irrelevant isotype controls.

Lung Parenchyma Cell Counts

Individual tissue sections immunohistochemically stained with one of CD4, CD8 (see above) were assessed for the number of positive cells in the parenchymal regions of the lung. Regions of intact lung parenchyma were visualized at 400× magnification using a microscope with graticule attachment. All positive cells within the boundaries of the graticule were counted and field of view was repositioned to a new area as necessary to obtain a count of at least 100 positive cells, recording both the number of fields and the total number of cells per sheep. The area of the graticule at 400× magnification was determined to be 0.078 mm², this was used to calculate the cell density (cells/area; data are presented as cells/mm²).

Collection of Bronchoalveolar Lavage

Bronchoalveolar (BAL) fluid was collected for analysis from all sheep from the respective lung segments. To collect BAL cells/fluid, a flexible fibre-optic bronchoscope was advanced into the selected lung segments and a lavage was collected by instillation and withdrawal of approximately 10 mL aliquots of PBS solution. The samples were placed immediately on ice. The cells were separated from the supernatant by centrifuging the lavage fluid for 7 min at 1000 rpm to remove cells. Supernatant was stored at −80° C. until use.

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Considering now the data, it is clear that in all three sheep the administration of bleomycin had a negative effect on lung function, increased inflammation and induced fibrosis, as compared to the sham treated right medial segment. Comparing data for the left and right caudal segments show that improvement in lung function and decrease in inflammation and fibrosis is attributable to the administration of pinocembrin. The beneficial effect of pinocembrin was statistically significant and noted in all three sheep.

### Example 2: Demonstration of Efficacy of 5,7-Dihydroxy-2-Phenyl-2,3-Dihydro-4H-Chromen-4-One (Pinocembrin) by Infusion into a Single Caudal Lung Segment in the Treatment of Inflammation and Fibrosis in a Sheep Pulmonary Model

Materials, Methods and Analyses

Induction of Fibrosis in Sheep Lung Segments

In this study, a total of 10 sheep were used. Fibrosis in two lung segments of each sheep in a similar manner as performed in Example 1 herein. Using this procedure (as detailed further below) the fibrosis was confined to small, isolated regions in the lungs leaving the remaining 90-95% of the healthy unaffected lungs to undertake normal respiratory function.

All sheep (n=10) in the study were challenged with 2 single doses of bleomycin (3 units) per lung segment, a fortnight apart and the animals were kept for a little over 5 weeks after the final bleomycin dose. Bleomycin is a well-known agent for inducing fibrosis in the lungs. The administration procedure involves inserting a bronchoscope into lung segments in the right and left lung, and then slowly infusing the bleomycin into two segments as shown in FIG. 2 via the bronchoscope biopsy port.

In the study detailed in Example 1 herein, the dose-rate of pinocembrin was 7 mg pinocembrin per sheep, per week. Note that this relatively low dose allowed for the fact that only a relatively small area in one lung-segment was treated, and not the whole lung, or the whole body, which would otherwise require significantly more drug for each sheep. As the study of Example 1 showed at 7 mg per sheep, it was decided to use the same dose-rate in this Example (i.e. 7 mg pinocembrin was infused into a single caudal lung segment in each sheep). The pinocembrin was dissolved in 10% DMSO, and given four times, one week apart, as shown in FIG. 1.

The administration procedure involved infusing the bioactive molecules in vehicle into the lung-segment as indicated in FIG. 1. For control purposes the lung segment in the opposite lung was used as a bleomycin-positive, without drug, control, as indicated in FIG. 1. The bioactive molecules in vehicle, were delivered as 5 ml infusions through the biopsy port of the bronchoscope. Note that to nullify any small differences (e.g. physiological or anatomical etc.) between the left and right lungs, the infusions of pinocembrin were randomized between the left and right caudal lung segments. Half the animals (5 sheep) received pinocembrin to the right caudal lung segments, while the other 5 sheep received pinocembrin to the left caudal lung segments. The right medial lung segment was left untreated and used for healthy lung control tissue which was sampled at autopsy (FIG. 2).

Bronchoalveolar Lavage Sampling Procedures

For each of the bronchoalveolar (BAL) samplings at the timepoints listed in FIG. 1, the endoscope was manipulated into a specific lung-segment for sampling, usually passing through about 3-4 airway branches. For BAL sampling, 10 ml of sterile saline was infused through the biopsy port of the endoscope into the specific lung-segment and then recovered into a syringe through the same port. This procedure recovered between 3 and 5 ml of BAL fluid. The sampling method collects cells for analyses from the small airway and alveolar lumens of the specific lung-segment where the bronchoscope was navigated to. The BAL cells from each segment were centrifuged onto glass slides and stained for differential cell counting of inflammatory cells with Hem-Quik.

Lung Function Testing and Analyses

Lung function was measured in the described lung-segments at the time points indicated in FIG. 1. Lung function was assessed in all test lung-segments (left and right caudal lobes, and medial lobe in each sheep). The functional capacity of the lung segments was measured through the endoscope using the procedure outlined in Example 1 herein. The lung function parameter assessed in this study is referred to as compliance in the lung segment (abbreviated to Cseg). In general, compliance is a measure of how easily it is to inflate the lung. A poorly compliant lung is referred to as a stiff lung, which is typically more difficult to inflate

Blood Sampling

The sheep were bled at the times indicated in FIG. 1 by sampling 10 ml of blood from the jugular vein into a tube containing heparin. The blood was processed for blood cell count analyses.

Sheep Euthanasia and Sampling of Lung Tissues for Analyses

Sheep were euthanized a little over 5 weeks after the last bleomycin dose (early Week 12), as indicated in FIG. 1. Following euthanasia, the lungs were removed, and targeted lung-segments identified and carefully dissected free from surrounding tissue. Individual segments were then inflated with a 1:1 mixture of Optimal Cutting Temperature Compound (OCT, ProSciTech Ltd Pty) and sterile PBS solution, under pressure of approximately 20 cm/H₂O. Several serial transverse sections of the inflated segment were fixed in 10% neutral-buffered formalin and processed in paraffin for histopathological assessment. Remaining lung slices were embedded in OCT and frozen in cryo-moulds on aluminium boats floating on liquid nitrogen for immunochemistry analyses.

Paraffin-embedded tissue sections (5 μm) were stained with haematoxylin and eosin Y (H&E) for general histology and with Masson's trichrome staining to identify changes to collagen content within the lung parenchyma.

Histopathology Scoring

Histopathology of the lung parenchyma was assessed using a semi-quantitative scoring system as outline in Example 1 herein. Briefly, histology slides were all blinded so that the assessor did not know the treatment groups. For each H&E stained section, 10 consecutive, non-overlapping fields at ×20 magnification were graded based on the scoring criteria for fibrosis, inflammation and overall pathology scores as outlined in Example 1 herein. The areas were selected away from large airways and major blood vessels. Scores from all ten fields were then averaged to give representative scores for the parameters assessed in the sectioned lung segment.

Analysis of Collagen Protein Content Using the Hydroxyproline Assay

The hydroxyproline assay was used to extrapolate the collagen content and concentration of each segment. Briefly, frozen lung tissues from each segment were lyophilized to dry weight, hydrolyzed in 6M HCl, and assessed for hydroxyproline content by measuring the absorbance of reconstituted samples (in 0.1M HCl) at 558 nm using a Beckman DU-64 spectrophotometer (Beckman Coulter Inc, Brea, Calif.). Hydroxyproline content was determined from a standard curve of trans-L-hydroxy-L-proline (Sigma-Aldrich).). Collagen content was extrapolated by multiplying the hydroxyproline measurements by 6.94 (based on hydroxyproline representing 14.4% of the amino acid composition of collagen in most tissues) and then expressed as a proportion of the dry weight tissue to yield collagen concentration (which was expressed as a percentage).

Analysis of Connective Tissue Content: Masson's Trichrome Assay

The degree of fibrosis was quantified by assessing the changes in overall connective tissue content within the parenchymal tissue using methods known to the skilled person. To perform this analysis, paraffin sections of sheep lung tissues were stained using a Masson's trichrome stain which stains connective tissue blue. Briefly, images of Masson's trichrome-stained lung section were captured using a digital camera linked to microscope and computer. Ten fields were randomly captured under ×400 magnification excluding large blood vessels and bronchi. The images were then analysed using Image-Pro® Plus (Version 6.3 for Windows, Media Cybernetics, Bethesda, Md., USA) using the ‘colour selector’ tool to measure the area of blue-stained tissues (collagen and other connective tissues) within each field of view. The values for each of the ten images were then averaged for each slide. The fraction of blue stained tissue area was expressed as a percentage per total field area (percentage of blue stained tissue area per total field area). Image capturing and analysis were performed in a blinded manner in coded slides.

Analysis of CD4+ and CD8+ T Cells in Lung Parenchyma

For this analysis, frozen tissue sections from the left and right caudal lobes, and right medial lobes of all 10 sheep were cut and mounted on glass slides.

Immunohistochemistry was performed on these frozen tissue sections using the indirect immunoperoxidase technique. Specific monoclonal antibodies against sheep cell surface molecules were used to identify CD8 and CD4 T-lymphocyte subpopulations. For cell counts, either 200 immunoperoxidase positive cells were counted in a maximum of 20 microscope fields (×400 magnification) using an area-calibrated grid, or a minimum of 20 microscope fields were counted for less frequent counts.

Results

Animal Health and the Safety of Pinocembrin Administration

Health checks were routinely performed throughout the treatment periods and throughout the trial until euthanasia. This was to ascertain that the pinocembrin treatment caused no untoward health issues, or side-effects, to the sheep undergoing the trial. All animals remained healthy throughout the pinocembrin treatment period (week 8 to week 12, FIG. 2). During this period the animals continued to gain weight in the expected normal range for these sheep (FIG. 11) and there were no otherwise adverse health events.

It was found that throughout the pinocembrin treatment period the heart, and respiratory rates were within normal ranges expected for sheep under these housing conditions. The core temperature readings were also within the normal range. Differential counts of blood leukocytes were within normal ranges at both sampling time points.

In summary, based on the clinical assessment criteria used, it was found that pinocembrin treatment caused no untoward health effects to all ten sheep undergoing the trial.

The Effects of Pinocembrin on Lung Function

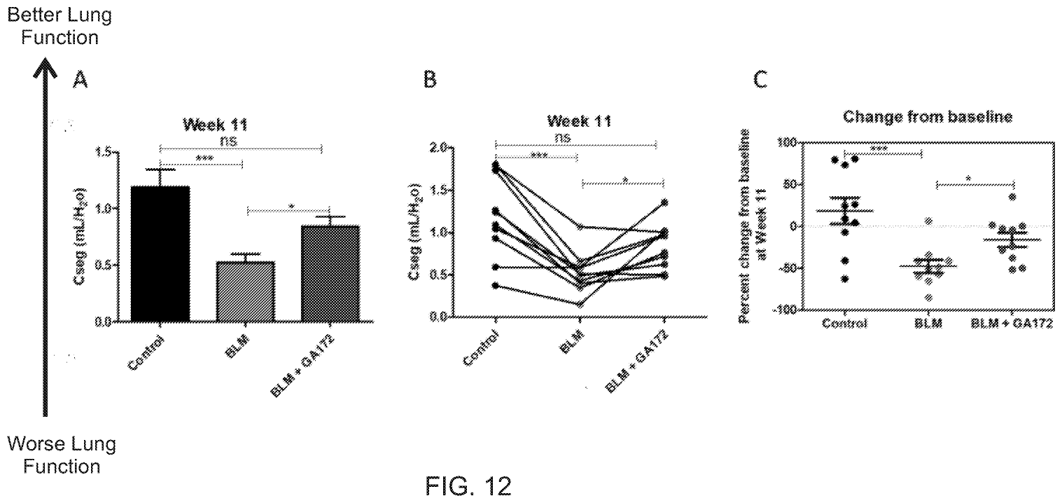

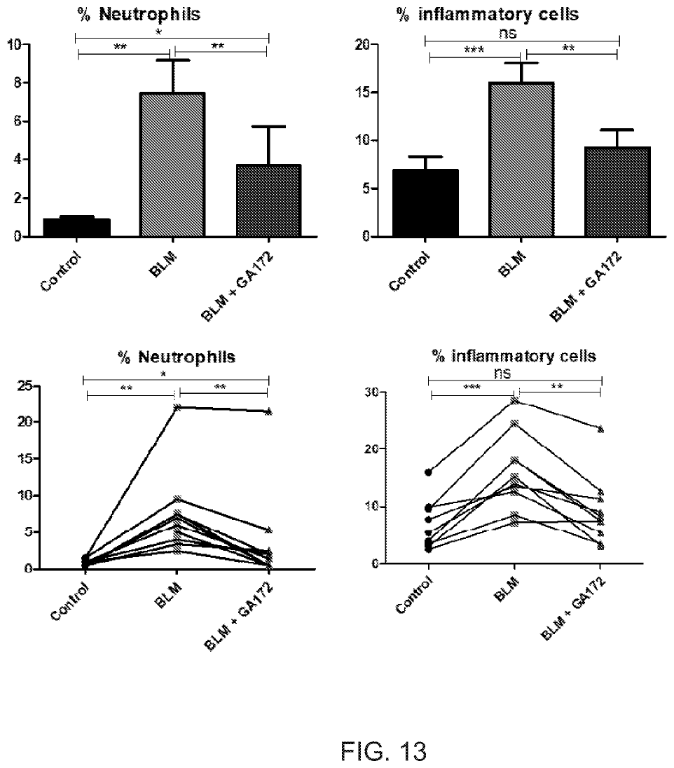

FIG. 12 shows lung function of the different lung-segments after four weekly treatments with pinocembrin. The lung function parameter measured was compliance in local lung segments and is referred to as Cseg. Generally, lower levels of compliance mean poorer function in the lung-segment (i.e. more difficult to inflate and the lung is stiffer). As expected, the lung-segments which received the damaging agent bleomycin alone, without pinocembrin, had significantly lower mean segmental compliance than the untreated healthy control lung-segments (FIG. 12A). The lung-segments, which received the damaging agent bleomycin with pinocembrin, had higher mean segmental compliance which is not significantly different from the untreated healthy control lung segments (FIG. 12A). Data for Cseg in individual sheep shows that eight out of ten sheep in the trial, had improved function in the lung-segments that were damaged with bleomycin and treated with four weekly infusions of pinocembrin (FIG. 12B, Table 1 as shown in FIG. 18). Another lung function assessment used was the percentage change in compliance from baseline (FIG. 12C). This measures the change in compliance from the start of the study (before bleomycin and pinocembrin treatments) to after the completion of the final pinocembrin treatment. The assessment showed that compliance in pinocembrin-treated lung segments had significantly improved after the four weekly administrations of pinocembrin (FIG. 12C).

In summary, pinocembrin treatment significantly improved the lung function in the lung segments injured by bleomycin.

The Effects of Pinocembrin on Bronchoalveolar Lavage Cells

FIG. 13 shows BAL cell data after four weekly infusion treatments with pinocembrin. The BAL cells were sampled from lung-segments during week 12 of the trial, two days before the sheep were culled. The cell counts assessed in the BAL fluid were neutrophils alone, and the sum of the main inflammatory cells, which included the neutrophils, eosinophils and lymphocytes. As expected for a normal healthy lung, the healthy control lung segments, which were untreated, had relatively low numbers of inflammatory cells in the BAL fluid (FIG. 13).

In contrast, the lung-segments injured by bleomycin, without pinocembrin, had significantly high numbers of neutrophils and other inflammatory cells in the BAL fluid compared to healthy control segments (FIG. 13). The lung-segments which received the damaging agent bleomycin, and had pinocembrin treatments, showed significantly lower numbers of neutrophils and inflammatory cells when compared to the cell numbers sampled from lung-segments, which received bleomycin alone without pinocembrin (FIG. 13). BAL cell data for individual sheep, shows that nine out of ten sheep participating in the trial, had lower inflammatory cell numbers that were sampled from lung segments that were damaged with bleomycin and treated with pinocembrin, when compared with inflammatory cell numbers in BAL fluid taken from lung segments that were injured with bleomycin without receiving pinocembrin infusions (FIG. 13, Table 1 as shown in FIG. 18).

In summary, pinocembrin treatment significantly reduced the number of inflammatory cells that infiltrate the BAL fluid in response to the damaging exposure of bleomycin. Nine out of ten sheep had reduced percentages of infiltrating inflammatory cells in the BAL fluid in these segments.

The Effect of Pinocembrin on Lung Parenchymal T Cells

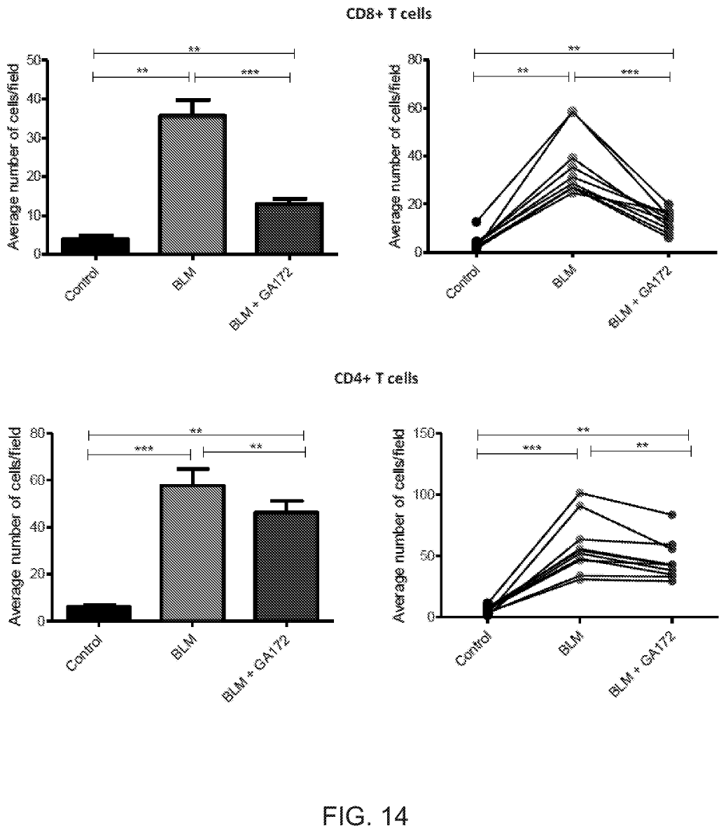

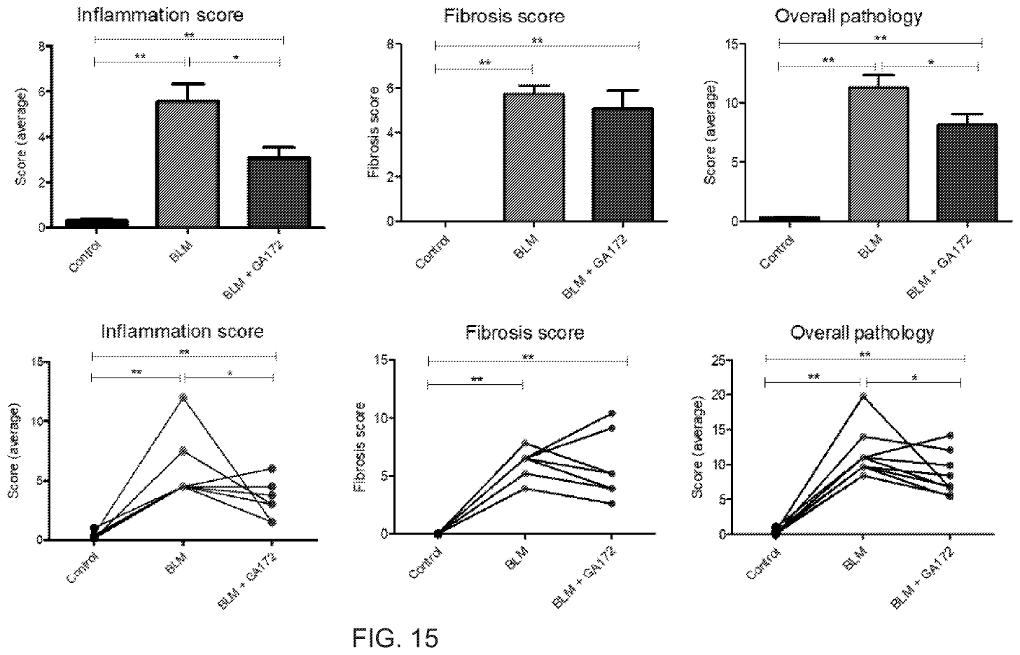

FIG. 14 shows T cell data after four weekly infusion treatments with pinocembrin. The T cells were assessed in the parenchyma of the differentially treated lung-segments which were sampled at post-mortem (week 12). As expected for a normal healthy lung, the healthy control lung segments, which were untreated, had relatively low numbers of CD8+ and CD4+ T cells in the lung parenchyma (FIG. 14).

In contrast, the lung-segments injured by bleomycin, without pinocembrin, had significantly higher numbers of CD8+ and CD4+ T cells in the lung parenchyma compared to healthy control segments (FIG. 14). The lung-segments which received the damaging agent bleomycin, and had pinocembrin treatments, showed significantly lower numbers of CD8+ and CD4+ T cells in the lung parenchyma when compared to the cell numbers sampled from lung-segments, which received bleomycin alone without pinocembrin (FIG. 14). Cell data for individual sheep, shows that all sheep participating in the trial, had lower CD8+ and CD4+ T cells after pinocembrin treatment (FIG. 14).

In summary, pinocembrin treatment was associated with a significant reduction in the number of immuno-stained CD8+ and CD4+ T cells residing in the lung parenchyma. All sheep assessed had reduced numbers of T cells in pinocembrin-treated lung segments.

The Effects of Pinocembrin on Histopathology

FIG. 15 shows histopathology scoring data after four weekly treatments with pinocembrin. The histopathology parameters scored were inflammation, fibrosis and overall pathology. As expected for a normal healthy lung, the healthy control lung-segments which were left untreated, had low scores for each pathology parameter assessed (FIG. 15). In contrast, the lung-segments injured by bleomycin, without pinocembrin, had significantly high mean scores for each parameter tested (FIG. 15). Importantly, the lung-segments, which received the injuring agent bleomycin, and had pinocembrin treatments, had lower mean scores for each parameter assessed (FIG. 15). Moreover, lung segments which received both bleomycin and pinocembrin had significantly lower inflammation and overall pathology scores compared to segments which received bleomycin infusion only (FIG. 15). While the lung segments which received bleomycin and pinocembrin infusion had lower fibrosis scores as compared to segments which received bleomycin infusion only, the difference was not statistically significant (FIG. 15). Histopathology data for individual sheep, shows that pinocembrin treatment was associated nine out of ten sheep participating in the trial having improved overall pathology scores, nine out of ten sheep having improved inflammation scores, and eight out of ten sheep having improved fibrosis scores (FIG. 15, lower panels, Table 1 as shown in FIG. 18). It should be noted that the significantly improved pathology scores associated with pinocembrin treatment were all still significantly higher for all three parameters assessed than the corresponding pathology scores for untreated control lung segments (FIG. 15).

In summary, pinocembrin treatment significantly improved histopathology scores for inflammation and overall pathology, in the lung segments injured by bleomycin. Nine out of ten sheep had improved overall pathology scoring in these segments.

The Effects of Pinocembrin on Collagen Concentration

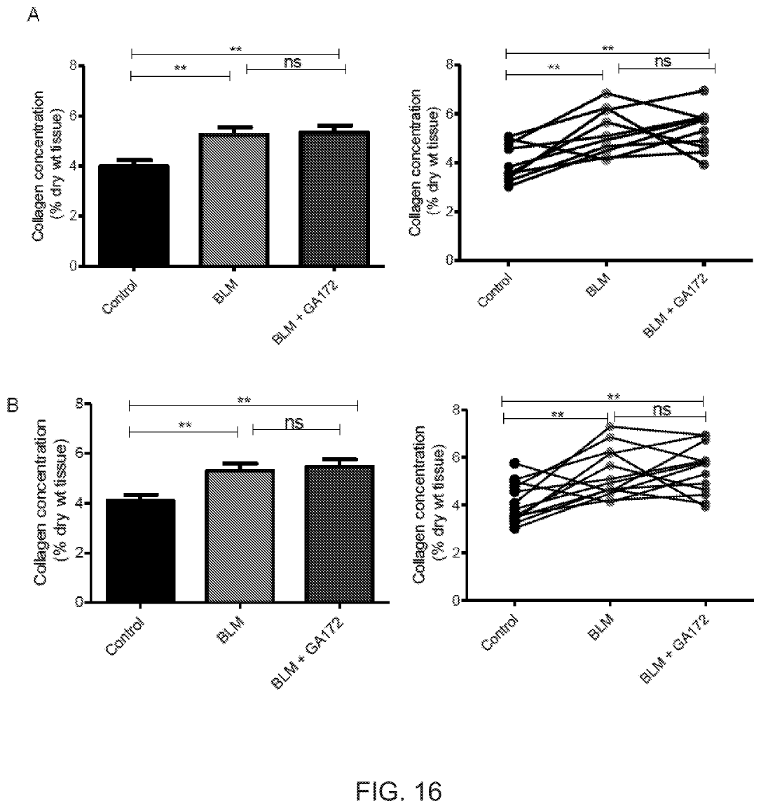

FIG. 16 shows data for the hydroxy proline assay for collagen content after four weekly treatments with pinocembrin. The data in FIG. 16A was collected from all 10 animals in the large trial and shows that bleomycin infusion alone (without pinocembrin) significantly increases collagen protein content compared with collagen data from healthy lung control segments which didn't receive either bleomycin, or pinocembrin. The administration of pinocembrin did not reduce the increased collagen content that was induced by bleomycin (FIG. 16A). To confirm these data, additional collagen content data from three sheep used in the trial study of Example 1 (see FIG. 16B) was included. The trial of Example 1 was conducted using an identical protocol to that used in this Example 2 trial. Thus, it was deemed scientifically acceptable to include hydroxy proline data from all 13 sheep for this assay. The data from all 13 sheep shown in FIG. 16B, reinforces the interpretation above given for FIG. 16A, namely, the administration of pinocembrin does not reduce the increased collagen content that was induced by bleomycin.

In summary, as assessed by the hydroxy proline assay, pinocembrin treatment was not associated with a significant decrease in collagen content in lung-segments injured by bleomycin.

The Effects of Pinocembrin on Masson's Trichrome-Stained Connective Tissue

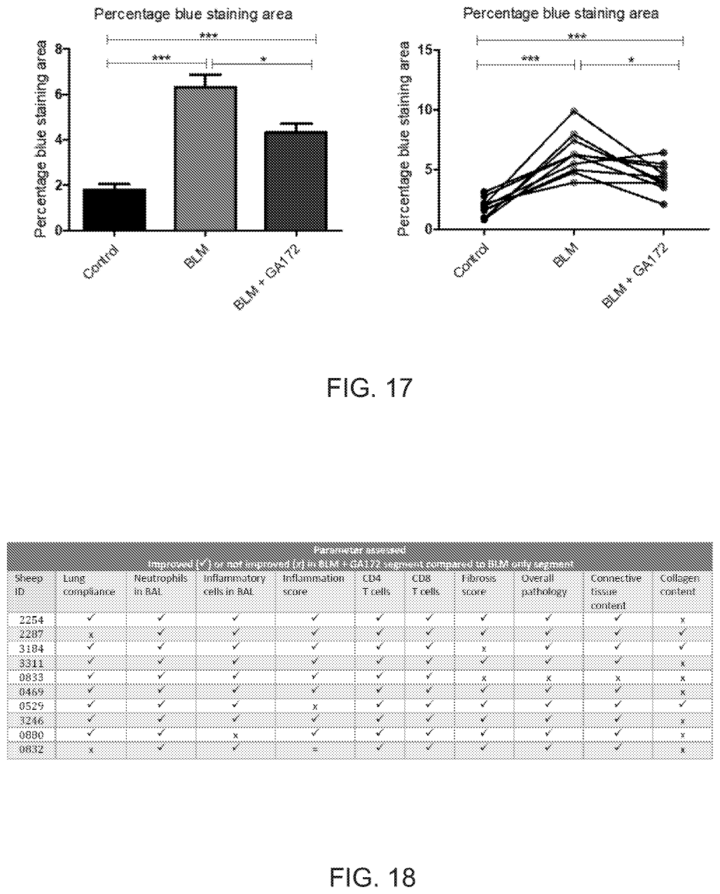

FIG. 17 shows data for Masson's Trichrome stained connective tissue after four weekly treatments with pinocembrin. Masson's Trichrome stains most connective tissues blue. The stain connective tissue includes collagen and other extracellular matrix proteins associated with fibrosis. Thus, the percentage blue value on Masson's trichrome sections is one readout for assessing the extent of fibrotic remodelling in bleomycin exposed lung segments. The data in FIG. 17 shows that pinocembrin treatment significantly reduces the percentage blue staining in lung segments exposed to bleomycin when compared to bleomycin-infused lung which did not receive pinocembrin treatment.

Overall, the data shows that nine out of ten sheep participating in the trial, had decreased percentage blue values in the lung-segments that were damaged with bleomycin and treated with four weekly infusions of pinocembrin, when compared with percentage blue scores associated with bleomycin injury without pinocembrin treatment (FIG. 1, Table 1 as shown in FIG. 18).

In summary, pinocembrin treatment was associated with a significant decrease in connective tissue content, as represented by percentage blue values in lung-segments injured by bleomycin.

Discussion

This study made use of a physiologically and pharmacologically relevant sheep model for pulmonary fibrosis to ascertain the safety, and signs of efficacy, of the bioactive molecule pinocembrin that was extracted by Gretals Australia Pty Ltd from specialised bio-sources.

The Health of Sheep Used in the Trial after Pinocembrin Treatment

In terms of the safety of pinocembrin treatment, based on the clinical assessment criteria used, it was found that pinocembrin treatment caused no untoward health effects in all ten sheep undergoing the trial. Indeed, throughout the pinocembrin treatment period, heart and respiratory rates, core temperatures, and weight gain readings, were within normal ranges expected for sheep in an animal house environment.

The small segment of lung that was exposed to pinocembrin was found to be relatively normal, with the only significant pathology being attributable to the expected residual effects of damage that was associated with bleomycin infusion. In the pinocembrin exposed segments there were no obvious signs of additional pathology or lung damage that could be attributable to pinocembrin.

The Efficacy of Pinocembrin to Ameliorate Several Disease Parameters in a Sheep Model of Experimental Lung Disease.

An aim of this study was to provide statistical power to the promising findings of the study detailed at Example 1 herein. In this Example, 10 sheep were used to statistically confirm the efficacy of pinocembrin in the sheep model of pulmonary fibrosis. The main findings were that the administration of pinocembrin was able to improve lung function, attenuate lung inflammation, and decrease the overall pathology scores which were induced by bleomycin injury. Importantly, the statistical analyses of the data revealed that these disease readouts were significantly improved in pinocembrin-treated lung segments when compared with the corresponding data from control non-treated lung segments.

In the case of lung function, as assessed by compliance (which is a measure of stiffness of the lung segment) it was found that pinocembrin treatment significantly improved functional compliance in the lung segments injured by bleomycin. This demonstrates that actions of pinocembrin treatment in injured lung segments results in those segments functioning at higher levels than otherwise would be.

Similarly, the identification of lung lavage inflammatory cells recovered from lung segments under investigation, showed that pinocembrin treatment significantly reduces the number inflammatory cells that occupy the luminal spaces of alveoli and small airways. Indeed, nine out of ten sheep had reduced numbers of inflammatory cells recovered from the damaged lung segments after pinocembrin treatment. The main inflammatory cell type that was reduced in the lung lavage fluid was the neutrophil which dropped from 7.4% of total BAL cells in the bleomycin without drug treatment lung segments to 3.7% in the pinocembrin-treated bleomycin-injured lung segments. The pinocembrin-associated reduction in CD4+ and CD8+ T cells in the lung parenchyma is entirely consistent with the reduction of inflammatory cells recovered from the lung lavage fluid of pinocembrin- and bleomycin-exposed lung segments. CD4+ and CD8+ T cells are important components in the cellular arms of many immune responses. Overall, these data support the notion that pinocembrin has strong anti-inflammatory properties.

In terms of histopathology scores, the mean inflammation and overall pathology scores were improved in the injured lung segments after pinocembrin treatment. Importantly, these readouts, were statistically lower in the pinocembrin-treated damaged lungs, compared to the experimentally injured lungs without pinocembrin treatment. Moreover, the mean fibrosis pathology scores were lower in the pinocembrin-treated damaged lungs, compared to the experimentally injured lung without pinocembrin treatment.

A hydroxyproline assay was performed on tissue samples from the differentially treated lung segments. The hydroxyproline assay measures the level of collagen in a protein sample and is considered in the art as a gold standard readout measure for the level of fibrosis in tissues. This assay showed that pinocembrin did not address the increase in collagen protein content associated with bleomycin injury. The Masson's trichrome assay (another readout measure that is frequently used to assess the extent of fibrosis) showed that the percentage blue staining (i.e. a measure of connective tissue content, all extracellular proteins stain blue in this assay) was significantly lower in bleomycin exposed and pinocembrin treated lung sections, when compared to bleomycin exposed lung sections which did not receive drug treatments. Taken together, data from both the hydroxy proline and Masson's trichrome assays, suggest that pinocembrin has the ability to reduce some extracellular matrix proteins (shown by Masson's trichrome data), but not necessarily collagen (as corroborated by the hydroxy proline data). Overall, the data from fibrosis scores, Masson's trichrome and hydroxy proline assays indicate that pinocembrin has a modest anti-fibrotic effect, and also some anti-remodelling properties.

Pinocembrin administration was started at day 7 after the final bleomycin infusion, which means that the drug was administered post-acute-inflammation, and predominantly in the fibrotic phase of pulmonary fibrosis. The fact that pinocembrin was administered in the fibrotic phase and showed modest anti-remodelling effects, but strong anti-inflammatory effects, gives confidence that pinocembrin should translate well for treating a range of human inflammatory diseases.

Those skilled in the art will appreciate that the invention described herein is susceptible to further variations and modifications other than those specifically described. It is understood that the invention comprises all such variations and modifications which fall within the spirit and scope of the present invention.

While the invention has been disclosed in connection with the preferred embodiments shown and described in detail, various modifications and improvements thereon will become readily apparent to those skilled in the art.

Accordingly, the spirit and scope of the present invention is not to be limited by the foregoing examples, but is to be understood in the broadest sense allowable by law.

1. A method for the treatment and/or prophylaxis of a fibrotic or inflammatory condition, the method comprising the administration of an effective amount of a flavonoid to an animal in need thereof.

2. The method of claim 1, wherein the fibrotic condition is caused at least in part by a wound healing response.

3. The method of claim 2, wherein the wound healing response occurs in a tissue comprising epithelial and/or endothelial cells.

4. The method of claim 1, wherein the fibrotic condition is selected from the group consisting of: pulmonary fibrosis (including idiopathic pulmonary fibrosis, infection-induced pulmonary fibrosis, radiation-induced pulmonary fibrosis, progressive massive fibrosis, cystic fibrosis), pancreatic fibrosis (including cystic fibrosis), retropertinoneal fibrosis, arterial fibrosis (including arterial stiffness), intestinal fibrosis (including Crohn's disease), joint fibrosis (including athrofibrosis of the knee, shoulder and other joints, adhesive capsulitis), manual/digital fibrosis (including Dupuytren's contracture), dermal fibrosis (including keloid, nephrogenic systemic fibrosis, scleroderma), penis (including Peyronie's disease), lymph node fibrosis (including mediastinal fibrosis) and myocardial fibrosis (including interstitial fibrosis and replacement fibrosis).

5. The method of claim 1, wherein the fibrotic condition is a pulmonary fibrosis, and the inflammatory condition is a pulmonary inflammation.

6. The method of claim 1, wherein the flavonoid is a flavanone.

7. The method of claim 1, wherein the flavanone has a chemical structure according to formula 1 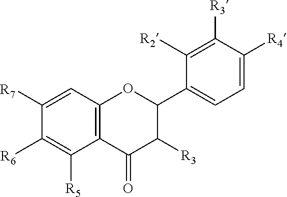
wherein R2′, R3, R3′, R4′, R5, R6, R7 are each independently:
H,
OH,
O—,
O—CH3,
a glucoside (including a rhamnosidoglucoside), or
any other organic functional group.

8. The method of claim 7, wherein R2′, R3, R3′, R4′, R5, R6, R7 are as follows:

| R3 | R5 | R6 | R7 | R2′ | R3′ | R4′ | Name |
|---|---|---|---|---|---|---|---|
| H | H | H | H | H | H | H | Flavanone |
| H | OCH3 | H | H | H | H | H | 5-Methoxyflavanone |
| H | H | OH | H | H | H | H | 6-Hydroxyflavanone |
| H | H | OCH3 | H | H | H | H | 6-Methoxyflavanone |
| H | H | H | OH | H | H | H | 7-Hydroxyflavanone |
| H | H | H | H | OH | H | H | 2′-Hydroxyflavanone |
| H | H | H | H | H | H | OH | 4′-Hydroxyflavanone |
| H | H | H | H | H | H | OCH3 | 4′-Methoxyflavanone |
| H | OH | H | OH | H | H | H | Pinocembrin |
| H | OH | H | OCH3 | H | H | H | Pinocembrin-7-methylether |
| H | OH | H | OH | H | H | OH | Naringenin |
| H | OH | H | OH | H | H | OCH3 | Isosakuranetin |
| H | OH | H | OCH3 | H | H | OH | Sakuranetin |
| H | OH | H | Gla | H | H | OH | Naringenin-7-glucoside |
| H | OH | H | Rh-Glb | H | H | OH | Naringin |
| H | OH | H | OH | H | OH | OH | Eriodictyol |
| H | OH | H | OH | H | OCH3 | OH | Homoeriodictyol |
| H | OH | H | OH | H | OH | OCH3 | Hesperetin |
| OH | OH | H | OH | H | OH | OH | Taxifolin |

a; Gl = Glucoside.
b; Rh-Gl = Rhamnosidoglucoside.

9. The method of claim 6, wherein the flavanone is dihydroxyflavanone and/or a (2S)-flavan-4-one, or a functional derivative thereof.

10. The method of claim 6, wherein the flavanone is (2S)-5,7-dihydroxy-2-phenyl-2,3-dihydrochromen-4-one, or a functional derivative thereof.

11. The method of claim 1, wherein the flavonoid is of the type naturally synthesized in a plant cell, although is not necessarily obtained from a plant cell for use in the method.

12. The method of claim 1, wherein use of the flavonoid in a sheep model of lung disease results in an improvement in any one or mode of lung function, presence of neutrophils and/or inflammatory cells in a lung lavage fluid, histologically assessed inflammation and/or fibrosis.

13. The method of claim 12, wherein the sheep model of lung disease relies on bleomycin-induced lung damage.

14. The method of claim 1, wherein the flavonoid is delivered directly to the tissue having fibrosis, potentially having fibrosis or predicted to have fibrosis in the future.

15. The method of claim 14, wherein the flavonoid is delivered directly to the lungs.

16. The method of claim 15, wherein the flavonoid is formulated as an inhalable powder or a solution deliverable by a nebulizer, or a solution deliverable by a bronchoscope.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A pharmaceutical composition comprising a flavonoid, the composition being formulated as an inhalable powder or a solution deliverable by a nebulizer, or a solution deliverable by a bronchoscope so as to be suitable for direct delivery to the lungs and/or airways of an animal, wherein the flavonoid is a flavanone having a chemical structure according to formula 1: 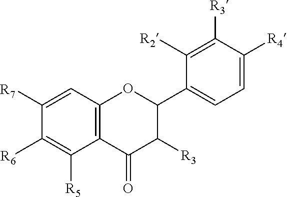
wherein R2′, R3, R3′, R4′, R5, R6, R7 are each independently;
H,
OH,
O—,
O—CH3,
a glucoside (including a rhamnosidoglucoside), or
any other organic functional group.

50. (canceled)

51. (canceled)

52. (canceled)

53. (canceled)

54. The pharmaceutical composition of claim 49, wherein R2′, R3, R3′, R4′, R5, R6, R7 are as follows:

| R3 | R5 | R6 | R7 | R2′ | R3′ | R4′ | Name |
|---|---|---|---|---|---|---|---|
| H | H | H | H | H | H | H | Flavanone |
| H | OCH3 | H | H | H | H | H | 5-Methoxyflavanone |
| H | H | OH | H | H | H | H | 6-Hydroxyflavanone |
| H | H | OCH3 | H | H | H | H | 6-Methoxyflavanone |
| H | H | H | OH | H | H | H | 7-Hydroxyflavanone |
| H | H | H | H | OH | H | H | 2′-Hydroxyflavanone |
| H | H | H | H | H | H | OH | 4′-Hydroxyflavanone |
| H | H | H | H | H | H | OCH3 | 4′-Methoxyflavanone |
| H | OH | H | OH | H | H | H | Pinocembrin |
| H | OH | H | OCH3 | H | H | H | Pinocembrin-7-methylether |
| H | OH | H | OH | H | H | OH | Naringenin |
| H | OH | H | OH | H | H | OCH3 | Isosakuranetin |
| H | OH | H | OCH3 | H | H | OH | Sakuranetin |
| H | OH | H | Gla | H | H | OH | Naringenin-7-glucoside |
| H | OH | H | Rh-Glb | H | H | OH | Naringin |
| H | OH | H | OH | H | OH | OH | Eriodictyol |
| H | OH | H | OH | H | OCH3 | OH | Homoeriodictyol |
| H | OH | H | OH | H | OH | OCH3 | Hesperetin |
| OH | OH | H | OH | H | OH | OH | Taxifolin |

a; Gl = Glucoside.
b; Rh-Gl = Rhamnosidoglucoside.

55. The pharmaceutical composition of claim 49, wherein the flavanone is dihydroxyflavanone and/or a (2S)-flavan-4-one, or a functional derivative thereof.

56. The pharmaceutical composition of claim 49, wherein the flavanone is (2S)-5,7-dihydroxy-2-phenyl-2,3-dihydrochromen-4-one, or a functional derivative thereof.

57. (canceled)

58. (canceled)

59. (canceled)
